SEQUENCE LISTING (6 sequences)

SEQ ID NO: 1 - protein, 465 aa, Artificial Sequence
  Synthetic
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 2 - protein, 293 aa, Artificial Sequence
  Synthetic
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKTRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 3 - DNA, 7733 bp, Artificial Sequence
  Synthetic
gatctgctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctattaattcaacattttgacacccccataatatttttccagaattaacagtataaattgcatctcttgttcaagagttccctatcactctctttaatcactactcacagtaacctcaactcctgccacaagcttgaatttcctggaggcttgctgaaggctgtatgctgaatgtaagctggcagaccttcgttttggccactgactgacgaaggtctcagcttacattcaggacacaaggcctgttactagcactcacatggaacaaatggcgcaagcggccgcgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcttacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacgatggcttctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttattaagaattcacgcgtcgagcatgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatggactatcctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagaggagtgcaggtggaaaccatctccccgggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaagtcgagggcgtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagttattaaggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgcaggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccta

SEQ ID NO: 4 - DNA, 2943 bp, Artificial Sequence
  Synthetic
atggcttctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttattaagaattcacgcgtcgagcatgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatggactatcctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagaggagtgcaggtggaaaccatctccccgggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaagtcgagggcgtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagttattaa

SEQ ID NO: 5 - DNA, 21 bp, Artificial Sequence
  Synthetic
taagcatggagctagcaggct

SEQ ID NO: 6 - DNA, 120 bp, Artificial Sequence
  Synthetic
acattttgacacccccataatatttttccagaattaacagtataaattgcatctcttgttcaagagttccctatcactctctttaatcactactcacagtaacctcaactcctgccacaa

(19) United States
(12) Patent Application Publication
(10) Pub. No.: US 2023/0089312 A1
(43) Pub. Date: Mar. 23, 2023

(54) INDUCIBLE SINGLE AAV SYSTEM AND USES THEREOF

(71) Applicant: University of Massachusetts, Boston, MA (US)

(72) Inventors: Christian Mueller, Concord, MA (US); Neil Aronin, Newtonville, MA (US)

(73) Assignee: University of Massachusetts, Boston, MA (US)

(21) Appl. No.: 17/801,917

(22) PCT Filed: Feb. 24, 2021
(86) PCT No.: PCT/US2021/019385

§ 371 Date: Aug. 24, 2022

Related U.S. Application Data
(60) Provisional application No. 62/981,484, filed on Feb. 25, 2020.

(51) Int. Cl. C12N 15/63; C12N 15/86; C12N 9/12

(52) U.S. Cl. CPC C12N 15/635; C12N 15/86; C12Y 207/11001; C12N 9/12; C12N 2310/141; C12N 2840/203; C12N 2750/14143

(57) ABSTRACT

Aspects of the disclosure relate to compositions and methods for regulation of transgene (e.g., miRNAs, shRNAs or coding sequences) expression from viral vectors. In some embodiments, the disclosure provides expression constructs comprising a viral vector encoding one or more transgenes, the expression of which is regulated by a rapamycin/rapalog-based system.

### RELATED APPLICATIONS

This application is a national stage filing under 35 U.S.C. § 371 of international PCT application PCT/US2021/019385, filed Feb. 24, 2021, which claims priority under 35 U.S.C. § 119(e) to U.S. provisional patent application, U.S. Ser. No. 62/981,484, filed Feb. 25, 2020, the entire contents of each of which are incorporated by reference herein.

Reference To A Sequence Listing Submitted As A Text File Via EFS-Web

This application contains a Sequence Listing which has been submitted in ASCII format via EFS-Web and is hereby incorporated by reference in its entirety. Said ASCII copy, created on Aug. 24, 2022, is named U0120.70129US01-SEQ-KZM and is 21,631 bytes in size.

### BACKGROUND

AAV mediated RNAi is at the forefront in the gene therapy revolution to target dominant genetic diseases. Given the expanded role that AAV mediated gene therapy has filled using DNA directed transcription of artificial microRNAs as a modality to silence offending gene transcripts, it is imperative that a clinically compatible system for microRNA regulation be developed. Historically gene regulation with rAAV vectors has mostly relied on the use of the tet operon, a bacterial based system responding to the presence of doxycycline as the small molecule needed for gene regulation. This promoter system has proven effective for gene regulation both as the “tet on” and “tet off” versions. However, the largest roadblock to this system is its reliance on the expression of foreign proteins of bacterial origin, which has been shown to be targeted for elimination by the immune system.

### SUMMARY

Aspects of the disclosure relate to methods and compositions for modulation of transgene expression in a cell or subject. The disclosure is based, in part, on expression constructs (e.g., isolated nucleic acids, vectors such as viral vectors, pharmaceutical compositions, etc.) comprising (i) a regulatable promoter which is responsive to rapamycin or rapalog that is operably linked to a transgene encoding a gene product, and (ii) an expression cassette encoding one or more proteins involved with rapamycin (or rapalog) binding fused to a transcriptional activator domain and/or a DNA binding protein that specifically binds to the regulatable promoter. Without wishing to be bound by any particular theory, compositions of the disclosure allow for tunable expression of one or more transgenes from a single vector based upon increase or decrease in the concentration of rapamycin or a rapalog.

In some aspects, the present disclosure provides an isolated nucleic acid comprising: (i) a first expression cassette comprising a regulatable promoter operably linked to a first transgene; and (ii) a second expression cassette comprising second promoter operably linked to a second transgene, wherein the second transgene encodes a FKBP-rapamycin binding (FRB) protein, a transcription activator domain, a rapamycin-binding protein (FKBP), and a DNA binding domain that specifically binds to a portion of the regulatable promoter sequence of (i).

In some embodiments, the regulatable promoter is a weak promoter. In some embodiments, the weak promoter is a IL-2 promoter. In some embodiments, the regulatable promoter can be any promoter that can be recognized by a zinc finger domain encoded by the isolated nucleic acid.

In some embodiments, the first transgene comprises a nucleotide sequence encoding an inhibitory nucleic acid, a transfer RNA (tRNA), a guide RNA, or an aptamer. In some embodiments, the inhibitory nucleic acid is an inhibitory RNA selected from the group consisting of a microRNA, a small interfering RNA (siRNA), a short hairpin RNA, an artificial miRNA (AmiRNA) or an antagomir. In some embodiments, the inhibitory RNA is an inhibitory RNA targeting alpha-1-antitrypsin (AAT), human huntingtin gene (HTT), Nav1.7, FAAH, or PCSK9. In some embodiments, the inhibitory RNA is an artificial miRNA targeting alpha-1-antitrypsin (AAT). In some embodiments, the small RNA is a miRNA targeting human huntingtin gene (HTT). In some embodiments, the miRNA targets exon 48 near position 6433 of the HTT gene. In some embodiments, the first transgene comprises a nucleotide sequence encoding a protein selected from the group consisting of a cytokine, a growth factor, a toxin, a minigene, a Fab, and a nanobody.

In some embodiments, the second promoter is a constitutive promoter, an inducible promoter, or a tissue specific promoter. In some embodiments, the second promoter is a minimal promoter.

In some embodiments, the FKBP is FKBP12. In some embodiments, the DNA binding domain is a zinc finger domain or a dCas protein. In some embodiments, the FKBP is fused to the DNA binding domain. In some embodiments, the FKBP is fused to the DNA binding protein via a linker, optionally wherein the linker is a polypeptide linker. In some embodiments, the FKBP12-zinc finger protein fusion protein comprises an amino acid sequence at least 80% identical to amino acid sequence of SEQ ID NO: 1.

In some embodiments, the FRB domain is a FKBP12-rapamycin-associated protein (FRAP) domain. In some embodiments, the transcription activator domain is p65 activation domain, VP4, or VP16. In some embodiments, the FRB domain is fused to the transcription activator domain. In some embodiments, the FRB is fused to the transcription activator domain via a linker, optionally wherein the linker is a polypeptide linker. In some embodiments, the FRB-p65 fusion protein comprises an amino acid sequence at least 80% identical to amino acid sequence of SEQ ID NO: 2.

In some embodiments, the isolated nucleic acid further comprises a IRES or a 2A peptide coding sequence, wherein the IRES or 2A is located between the FKBP12-zinc finger protein fusion protein and the FRB-p65 fusion protein.

In some embodiments, the first and/or the second transgene each further comprises a 3′ untranslated region (3′UTR). In some embodiments, the first or the second transgene each further comprises one or more miRNA binding sites. In some embodiments, the one or more miRNA binding sites are positioned in a 3′UTR of the first transgene. In some embodiments, the at least one miRNA binding site is an immune cell-associated miRNA binding site, or a liver-cell associated miRNA binding site. In some embodiments, the immune cell-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152. In some embodiments, the liver cell-associated miRNA is miR-122.

In some embodiments, the isolated nucleic acid further comprising two flanking long terminal repeats (LTRs).

In some embodiments, the isolated nucleic acid further comprising two flanking adeno-associated virus inverted terminal repeats (ITRs). In some embodiments, the ITRs are adeno-associated virus ITRs of a serotype selected from the group consisting of AAV1 ITR, AAV2 ITR, AAV3 ITR, AAV4 ITR, AAV5 ITR, and AAV6 ITR.

In some embodiments, the isolated nucleic acid is a closed-ended linear duplex DNA (ceDNA).

In some aspects, the present disclosure provides a vector comprising the isolated nucleic acid. In some embodiments, the vector is a plasmid, a lentiviral vector, a retroviral vector, an anellovirus vector, or an adeno-associated virus vector. In some embodiments, the vector comprises a nucleic acid sequence at least 80% identical to the nucleic acid sequence of SEQ ID NO: 3.

In some aspects, the present disclosure provides a recombinant adeno-associated virus (rAAV) vector comprising, in 5′ to 3′ order: (a) a 5′ AAV ITR; (b) a regulatable promoter; (c) a first transgene; (d) a second promoter; (e) a second transgene comprising a nucleotide sequence encoding a FRB-p65 fusion protein, a nucleotide sequence encoding an internal ribosome entry site (IRES), a nucleotide sequence encoding a FKBP-zinc finder binding domain fusion protein; and (f) a 3′ AAV ITR.

In some aspects, the present disclosure provides a recombinant lentivirus comprising a lentiviral capsid containing the isolated nucleic acid described herein.

In some aspects, the present disclosure provides a recombinant adeno-associated virus (rAAV) comprising: (i) an isolated nucleic acid described herein; and (ii) at least one AAV capsid protein. In some embodiments, the capsid protein is of a serotype selected from AAV1, AAV2, AAV3, AAV4, AAV5, AAV6, AAV7, AAV8, AAV9 and a variant of any of the foregoing. In some embodiments, the rAAV is a single-stranded AAV (ssAAV).

In some aspects, the present disclosure provides a recombinant adeno-associated virus (rAAV), comprising: (i) an AAV capsid protein; and (ii) an isolated nucleic acid comprising, in 5′ to 3′ order: (a) a 5′ AAV ITR; (b) a regulatable promoter; (c) a first transgene; (d) a second promoter; (e) a second transgene comprising a nucleotide sequence encoding a FRB-p65 fusion protein, a nucleotide sequence encoding an internal ribosome entry site (IRES), a nucleotide sequence encoding a FKBP-zinc finder binding domain fusion protein; and (f) a 3′ AAV ITR.

In some aspects, the present disclosure provides a host cell comprising: (i) isolated nucleic acid and the vector described herein, the recombinant lentivirus described herein, or the rAAV described herein; and (ii) rapamycin or a rapalog. In some embodiments, the host cell is a mammalian cell, yeast cell, bacterial cell, or insect cell. In some embodiments, the concentration of the rapalog is 10 nM to 2000 nM.

In some aspects, the present disclosure provides a pharmaceutical composition comprising the isolated nucleic acid, the vector, the recombinant lentivirus, the rAAV, or the host cell described herein. In some embodiments, the pharmaceutical further comprises a pharmaceutically acceptable carrier. In some embodiments, the pharmaceutical composition is formulated for intravenous injection, intraperitoneal injection, intracranial injection, intratumoral injection, intramuscular injection, or intravitreal injection.

In some aspects, the present disclosure provides a method for treating a disease in a subject in need thereof, the method comprising: (i) administering to the subject a therapeutically effective amount of the isolated nucleic acid, the vector, the recombinant lentivirus, the rAAV, the host cell or the pharmaceutical composition as described herein; and (ii) administering to the subject an effective amount of rapamycin or rapalog.

In some aspects, the present disclosure provides a method for treating an alpha1-antitrypsin associated disorder in a subject in need thereof, the method comprising: (i) administering to the subject a therapeutically effective amount of the isolated nucleic acid, the vector, the recombinant lentivirus, the rAAV, the host cell or the pharmaceutical composition as described herein; and (ii) administering to the subject an effective amount of rapamycin or rapalog. In some embodiments, the method further comprises supplementing the subject with wild-type ATT. In some embodiments, the isolated nucleic acid encodes an inhibitory RNA (e.g., short interfering RNA) targeting AAT.

In some aspects, the present disclosure provides a method for treating Huntington's disease in a subject in need thereof, the method comprising: (i) administering to the subject a therapeutically effective amount of the isolated nucleic acid, the vector, the recombinant lentivirus, the rAAV, the host cell or the pharmaceutical composition as described herein; and (ii) administering to the subject an effective amount of rapamycin or rapalog. In some embodiments, the isolated nucleic acid encodes a miRNA targeting human huntingtin gene (HTT). In some embodiments, the subject is a non-human mammal.

In some embodiments, the non-human mammal is mouse, rat, cat, dog, sheep, rabbit, horse, cow, goat, pig, guinea pig, hamster, chicken, turkey, or a non-human primate. In some embodiments, the subject is a human.

In some embodiments, the administration is intravenous injection, intraperitoneal injection, intracranial injection, intratumoral injection, intramuscular injection, or intravitreal injection. In some embodiments, administering (i) and (ii) is concurrent. In some embodiments, administering (i) and (ii) is sequential. In some embodiments, administering (i) and (ii) is at different frequencies. In some embodiments, (i) is administered once and (ii) is administered repeatedly. In some embodiments, (ii) is administered every week, every two weeks, or every month. In some embodiments, the dose of rapamycin or rapalog does not induce immunosuppression in the subject. In some aspects, the present disclosure provides a method for modulating transgene expression in a subject, the method comprising: (i) administering to the subject a therapeutically effective amount of the isolated nucleic acid, the vector, the recombinant lentivirus, the rAAV, the host cell or the pharmaceutical composition as described herein; (ii) measuring expression of transgene in the subject relative to a control expression level of the first transgene; (iii) adjusting the dose of rapalog based on expression level measured in (ii); wherein if the expression level measured in (ii) is increased relative to the control level, administering the same or less concentration of the rapalog; and wherein if the expression level measured in (ii) is the same or decreased relative to the control level, administering a higher concentration of the rapalog to the subject.

### BRIEF DESCRIPTION OF DRAWINGS

FIG. 1 shows the design of one embodiment of a rapamycin/rapalog inducible AAV vector.

FIG. 2A-2B are schematic designs of a rapalog regulatable system. FIG. 2A shows the rapalog-regulatable system which comprises two Rapa binding domains (FKBP and FRB). The FRP domain is fused to p65 which acts as transcription activator. The FKBP domain is fused to a zinc finger nuclease engineered to specifically bind the regulated promoter sequence. In the presence of ligand, the domains are brought together as a functional transcriptional unit at the regulated promoter sequence. The graph on the right shows the activation of miRNA transcription from the regulated promoter. Hek-293 cell were transfected with the regulated miR construct and expose to rapalog or PBS in the media. Cell RNA lysates were assayed for the presence of mature miRNA 48 hours after ligand exposure. The negative controls are cell that were not transfected. FIG. 2B are graphs showing how the rapalog regulatable system behave in the cells in the absence (left panel) or presence (right panel) of the heterodimerizer (e.g., rapalog). In these systems, the small molecule or “dimerizer” causes heterodimerization of two proteins comprising an activation domain and a DNA binding domain. The heterodimerization of these proteins leads to reconstitution of an active transcription factor, which induces expression of an miRNA. Left panel shows when no addition of A/C Heterodimerizer (−), transcription is not induced. Right panel shows the addition of A/C Heterodimerizer (+) leads to transcription.

FIG. 3 depicts microRNAs expression induced by Rapalog, showing dose-dependent miRNA induction by final concentration of Rapalog application.

FIG. 4 shows that microRNA expression decreases after Rapalog application is stopped.

FIG. 5 shows the effect of knock-down using Rapa-regulated miRNA against transgenic AAT.

FIG. 6 is a schematic drawing of a regulated mir-HTT6433 expression construct.

FIG. 7 depicts Mir-HTT6433 expression induced by Rapalog, showing miRNA expression induced by a series final concentration of Rapalog in cell culture medium.

FIG. 8 illustrates a luciferase assay, which shows dose-dependent knockdown of the Ff-luc-HTT-ex48 48 hours after rapalog application.

FIG. 9 shows human mutant HTT levels detected by western blot assay after two weeks of rapalog-regulation in AAV9-RAPA-miHTT treated BAC97 mice (RAPA+n=4), compared to AAV9-RAPA-miHTT treated BAC97 mice without rapalog-regulation (RAPA-n=5). A control group of BAC97 mice treated with non-regulated AAV9-miRHTT to compare efficacy with rapalog-mediated regulation (n=4). All groups are normalized to the PBS control group (n=3). Results are shown for the striatal region on the injected side of the brain. This graph shows distribution of individual animals and means (horizontal bars) for each treatment group.

FIGS. 10A-10H are charts showing the evaluation of AAV9-RAPA-miHTT in YAC128 mouse model. FIG. 10A shows the YAC128-HD Mouse Treatment Timeline. FIGS. 10B-10E show human mutant HTT levels detected by western blot assay after one month of rapalog-mediated regulation in AAV9-RAPA-miHTT treated YAC128 mice (RAPA+n=8), compared to AAV9-RAPA-miHTT treated YAC128 mice without rapalog-regulation (RAPA-n=5). A control group of YAC128 mice treated with non-regulated AAV9-miRHTT to compare efficacy with rapalog-mediated regulation (n=6). All groups are normalized to the PBS control group (n=3). FIG. 10B shows distribution HTT of individual animals and means (horizontal bars) for each treatment group for the striatal region on one side of the brain. FIG. 10C shows distribution HTT of individual animals and means (horizontal bars) for each treatment group for the cortex region on one side of the brain. Significance values are based on One-way ANOVA statistical analysis. FIGS. 10D-10E are western blot images show mutant HTT detected with AB 1 antibody and Vinculin in Striatal (FIG. 10D) and cortex (FIG. 10E). FIGS. 10F-10G are charts showing human mutant HTT mRNA levels measured by Droplet Digital PCR (DD PCR). Striatum HTT mRNA (FIG. 10F) and cortex HTT mRNA (FIG. 10G) distribution of individual animals was graphed and means (horizontal bars) for each treatment group. FIG. 10H are images showing immunohistochemistry brain sections stains for HTT on one side of the brain.

### DETAILED DESCRIPTION

Aspects of the disclosure relate to compositions and methods for modulating transgene expression from vectors (e.g., viral vectors) in a cell or subject. In some embodiments, the vectors comprise expression constructs encoding (i) a transgene operably linked to a regulatable promoter that is responsive to rapamycin/rapalog concentration, and (ii) one or more rapamycin binding proteins which are fused to a transcriptional activator domain and/or a DNA binding domain that specifically binds to the regulatable promoter. The disclosure is based, in part, modulation of transgene expression in a cell by varying a concentration of rapamycin or a rapalog in a cell containing such an expression vector.

In some aspects, the disclosure relates to isolated nucleic acids encoding one or more transgenes. An isolated nucleic acid, in some embodiments, comprises: (i) a first expression cassette comprising a regulatable promoter sequence operably linked to a first transgene; and (ii) a second expression cassette comprising second promoter operably linked to a second transgene, wherein the second transgene encodes a FKBP-rapamycin binding (FRB), a transcription activator domain, a rapamycin-binding protein (FKBP), and a DNA binding protein that specifically binds to a portion of the regulatable promoter sequence of (i).

### Regulatable Trans Gene Constructs

The disclosure relates, in some aspects, to isolated nucleic acids comprising an expression cassette encoding one or more transgenes. A “nucleic acid” sequence refers to a DNA or RNA sequence. In some embodiments, proteins and nucleic acids of the disclosure are isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.). In some embodiments, a nucleic acid encoding a complement control protein or portion thereof is codon-optimized (e.g., codon-optimized for expression in human cells).

In some embodiments, an isolated nucleic acid comprises a first expression cassette. In some embodiments, the first expression cassette comprises a regulatable promoter operably linked to a first transgene.

A regulatable promoter, as used herein, refers to a promoter that can be regulated by an input signal (e.g., regulated in response to a concentration of a small molecule, nucleic acid, protein etc.). In some embodiments, the regulatable promoter comprises a sequence recognizable by the DNA binding protein expressed by another transgene encoded by the same vector (e.g., a second transgene). Non-limiting examples of a regulatable promoters include inducible promoters (e.g., Tet-on promoter, Tet-off promoter, etc.), tissue specific promoters, promoters comprising binding sequences to a transcription factor, etc. In some embodiments, the regulatable promoter is activated by (e.g., expression of the first transgene is increased in response to) the presence of rapamycin or a rapalog.

Rapamycin, also referred to as sirolimus, is a macrolide compound produced by the bacteria Streptomyces hygroscopicus, which functions as an immunosuppressant and antifungal agent. In mammals, rapamycin binds to the mammalian target of rapamycin (mTOR), a serine/threonine-specific protein kinase that regulates cellular metabolism, growth, and proliferation. In some embodiments, rapamycin is used in combination with fusion constructs containing a rapamycin-binding FRB domain and an FKBP domain in order to mediate chemically-induced dimerization of the constructs, resulting in an inducible gene expression system. Such a system is described, for example, by Rivera et al. (1996) Nature Medicine. 2 (9): 1028-32. In some embodiments, a regulatable promoter is activated by (e.g., binds to) rapamycin or a rapalog (a derivative of rapamycin). Examples of rapalogs include but are not limited to temsirolimus, everolimus, and ridaforolimus. In some embodiments, a regulatable promoter is activated by (e.g., binds to) an ATP-competitive mTOR kinase inhibitor (e.g., a second generation mTOR inhibitor).

In some embodiments, a regulatable promoter is a weak promoter. A weak promoter, as used herein, refers to a promoter that comprises less consensus sequences that match that of a RNA polymerase, thereby having low affinity to the RNA polymerase. A weak promoter gives low transcription and thus low levels of protein product. Non-limiting examples of a weak promoter include IL-2 promoter, or lac repressor promoter. In some embodiments, the regulatable promoter is an IL-2 promoter. An exemplary nucleic acid sequence for IL-2 promoter is set forth in SEQ ID NO: 6:

ACATTTTGACACCCCCATAATATTTTTCCAGAATTAACAGTATAAATTG
CATCTCTTGTTCAAGAGTTCCCTATCACTCTCTTTAATCACTACTCACA
GTAACCTCAACTCCTGCCACAA

In some embodiments, the IL-2 promoter comprises a sequence recognizable by the DNA binding domain expressed by the second expression cassette.

In some embodiments, a regulatable promoter (e.g., IL-2 promoter) drives the expression of the first transgene. In some embodiments, the first transgene comprises a nucleic acid encoding an inhibitory nucleic acid. In some embodiments, the first transgene comprises a nucleic acid encoding an aptamer. In some embodiments, the inhibitory nucleic acid is an inhibitory RNA. Non-limiting examples of an inhibitory RNA include microRNA (miRNA), a short interfering RNA (siRNA), a short hairpin RNA (shRNA), an artificial miRNA (AmiRNA) or an antagomir.

Inhibitory RNAs are useful for translational repression and/or gene silencing via the RNAi pathway. Due to having a common secondary structure, hairpin-forming RNA share the characteristic of being processed by the proteins Drosha and Dicer prior to being loaded into the RNA-induced silencing complex (RISC). Duplex length amongst hairpin-forming RNA can vary. In some embodiments, a duplex is between about 19 nucleotides and about 200 nucleotides in length. In some embodiments, a duplex is between about between about 14 nucleotides to about 35 nucleotides in length. In some embodiments, a duplex is between about 19 and 150 nucleotides in length. In some embodiments, an inhibitory RNA has a duplex region that is 19, 20, 21, 22, 23, 24, 25, 26, 27, 28, 29, 30, 31, 32, or 33 nucleotides in length. In some embodiments, a duplex is between about 19 nucleotides and 33 nucleotides in length. In some embodiments, a duplex is between about 40 nucleotides and 100 nucleotides in length. In some embodiments, a duplex is between about 60 and about 80 nucleotides in length.

In some embodiments, nucleic acids are provided herein that contain or encode the target recognition and binding sequences (e.g., a seed sequence or a sequence complementary to a target) of any one of the inhibitory RNAs (e.g., shRNA, miRNA, AmiRNA) disclosed herein. A microRNA (miRNA) is a small non-coding RNA found in plants and animals and functions in transcriptional and post-translational regulation of gene expression. An artificial miRNA, as used herein, is derived by modifying native miRNA to replace natural targeting regions of pre-mRNA with a targeting region of interest. For example, a naturally occurring, expressed miRNA can be used as a scaffold or backbone (e.g., a pri-miRNA scaffold), with the stem sequence replaced by that of an miRNA targeting a gene of interest. An artificial precursor microRNA (pre-amiRNA) is normally processed such that one single stable small RNA is preferentially generated. In some embodiments, scAAV vectors and scAAVs described herein comprise a nucleic acid encoding an AmiRNA. In some embodiments, the pri-miRNA scaffold of the AmiRNA is derived from a pri-miRNA selected from the group consisting of pri-MIR-21, pri-MIR-22, pri-MIR-26a, pri-MIR-30a, pri-MIR-33, pri-MIR-122, pri-MIR-375, pri-MIR-199, pri-MIR-99, pri-MIR-194, pri-MIR-155, and pri-MIR-451.

The following non-limiting list of miRNA genes, and their homologues, which are also useful in certain embodiments of the vectors provided herein: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-7i, hsa-let-7i*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-12′7-3p, hsa-miR-1274a, hsa-miR-1274b, hsa-miR-1275, hsa-miR-12′7-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-29b-1*, hsa-miR-29b-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-33′7-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-548b-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-5481, hsa-miR-548m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*. In some embodiments, the above miRNAs may be encoded for in a vector provided herein (e.g., in a hairpin nucleic acid that replaces a mutant ITR). In some embodiments, sequences of the foregoing miRNAs may be useful as scaffolds or as targeting regions.

The inhibitory nucleic acid can be designed to target any gene of interest. Non-limiting examples proteins can be targeted by the inhibitory RNA include alpha-1-antitrypsin (AAT), human huntingtin gene (HTT), SCN9A, Fatty acid amide hydrolase (FAAH), or proprotein convertase subtilisin/kexin type 9 (PCSK9).

In some embodiments, the inhibitory RNA is an AmiRNA targeting alpha-1 anti-trypsin (ATT).

In some embodiments, the inhibitory RNA is an miRNA targeting human huntingtin gene (HTT). In some embodiment, the miRNA targets exon 48 near position 6433 of the HTT gene. In some embodiments, the miRNA targeting human HTT is miR-6433-anti-HTT, which is encoded by a nucleic acid comprising the nucleotide sequence

(SEQ ID NO: 5)
TAAGCATGGAGCTAGCAGGCT.

In some embodiments, the first transgene encodes a transfer RNA (tRNA). A tRNA, as used herein, refers an RNA molecule, typically 76 to 90 nucleotides in length, that serves as the physical link between the mRNA and the amino acid sequence of proteins. Transfer RNA does this by carrying an amino acid to the protein synthetic machinery of a cell called the ribosome. Complementation of a 3-nucleotide codon in a messenger RNA (mRNA) by a 3-nucleotide anticodon of the tRNA results in protein synthesis based on the mRNA code. As such, tRNAs are a necessary component of translation, the biological synthesis of new proteins in accordance with the genetic code. Exogenous delivery of tRNA has been previously described, e.g., Mirande, Processivity of translation in the eukaryote cell: Role of aminoacyl-tRNA synthetases, FEBS Letters, Volume 584, Issue 2, 21 Jan. 2010, Pages 443-447; and Krogager et al., Labeling and identifying cell-specific proteomes in the mouse brain, Nature Biotechnology volume 36, pages 156-159(2018).

In some embodiments, the first transgene encodes a guide RNA (gRNA). The terms “guide RNA” and “gRNA” are used in DNA editing involving CRISPR/Cas9 system. gRNA has also been described to engage with deaminases for programmable RNA editing, e.g., Qu et al., Programmable RNA editing by recruiting endogenous ADAR using engineered RNAs, Nat Biotechnol. 2019 September; 37(9); and Merkle et al., Precise RNA editing by recruiting endogenous ADARs with antisense oligonucleotides, Nat Biotechnol. 2019 February; 37(2):133-138. In some embodiments, the first transgene comprises a nucleotide sequence encoding a protein. It is within the scope of the present disclosure such that the first transgene can encode any protein of interest. Non-limiting examples of the proteins are cytokines, growth factors, toxins, minigenes, Fab fragments of an antibody, or nanobodies.

In some embodiments, the first transgene encodes a cytokine. Non-limiting examples of a cytokine include: TNFα, IFN-γ, IFN-α, TGF-β, IL-1, IL-2, IL-4, IL-10, IL-13, IL-17, IL-18, and chemokines. Chemokines are useful for studies investigating response to infection, immune responses, inflammation, trauma, sepsis, cancer, and reproduction, among other applications. Chemokines are known in the art, and are a type of cytokines that induce chemotaxis in nearby responsive cells, typically of white blood cells, to sites of infection. Non-limiting examples of chemokines include, CCL14, CCL19, CCL20, CCL21, CCL25, CCL27, CXCL12, CXCL13, CXCL-8, CCL2, CCL3, CCL4, CCL5, CCL11, and CXCL10.

In some embodiments, the first transgene encodes a growth factor. As used herein, the term “growth factors” refers to a naturally occurring substance capable of signaling between cells and stimulating cellular growth. Non-limiting examples of growth factors include Adrenomedullin (AM), Angiopoietin (Ang), Autocrine motility factor, Bone morphogenetic proteins (BMPs), Ciliary neurotrophic factor (CNTF), Leukemia inhibitory factor (LIF), Interleukin-6 (IL-6), Macrophage colony-stimulating factor (m-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B 1, Ephrin B2, Ephrin B3, Erythropoietin (EPO), Fibroblast growth factor 1(FGF1), Fibroblast growth factor 2(FGF2), Fibroblast growth factor 3(FGF3), Fibroblast growth factor 4(FGF4), Fibroblast growth factor 5(FGF5), Fibroblast growth factor 6(FGF6), Fibroblast growth factor 7(FGF7), Fibroblast growth factor 8(FGF8), Fibroblast growth factor 9(FGF9), Fibroblast growth factor 10(FGF10), Fibroblast growth factor 11(FGF11), Fibroblast growth factor 12(FGF12), Fibroblast growth factor 13(FGF13), Fibroblast growth factor 14(FGF14), Fibroblast growth factor 15(FGF15), Fibroblast growth factor 16(FGF16), Fibroblast growth factor 17(FGF17), Fibroblast growth factor 18(FGF18), Fibroblast growth factor 19(FGF19), Fibroblast growth factor 20(FGF20), Fibroblast growth factor 21(FGF21), Fibroblast growth factor 22(FGF22), Fibroblast growth factor 23(FGF23), Fetal Bovine Somatotrophin (FBS), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukin-1 (IL-1),IL-2,IL-3, IL-4, IL-5, IL-6, IL-7, Keratinocyte growth factor (KGF), Migration-stimulating factor (MSF), Macrophage-stimulating protein (MSP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-β), Tumor necrosis factor-alpha (TNF-α), and Vascular endothelial growth factor (VEGF).

In some embodiments, the first transgene encodes a toxin. In some embodiments, the first transgene encodes a minigene. In some embodiments, the first transgene encodes a nanobody. In some embodiments, the first transgene encodes a Fab fragment of an antibody. In some embodiments, the transgene encodes a minibody. In some embodiments, the transgene encodes a nanobody. In some embodiments, the first transgene encodes a single-chain variable fragment (scFv).

Additionally, the isolated nucleic acid further comprises a second expression cassette. In some embodiments, the second expression cassette comprises a second promoter operably linked to a second transgene. In some embodiments, a second expression cassette comprises a second promoter operably linked to a second transgene, wherein the second transgene encodes a FKBP-rapamycin binding (FRB), a transcription activator domain, a rapamycin-binding protein (FKBP), and a DNA binding protein that specifically binds to a portion of the regulatable promoter sequence of which is operably lined to the first transgene.

In some embodiments, a second promoter is a constitutive promoter, inducible promoter, or a tissue-specific promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the chimeric cytomegalovirus chimeric cytomegalovirus (CMV)/Chicken β-actin (CB) promoter (CBA promotor), the SV40 promoter, the dihydrofolate reductase promoter, the (β-actin promoter, the phosphoglycerol kinase (PGK) promoter, and the EF1α promoter [Invitrogen]. In some embodiments, a promoter is an RNA pol II promoter. In some embodiments, a promoter is the chimeric cytomegalovirus chimeric cytomegalovirus (CMV)/Chicken β-actin (CB) promoter (CBA promoter). In some embodiments, a promoter is an RNA pol III promoter, such as U6 or H1.

Examples of inducible promoters regulated by exogenously supplied signals include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: retinoschisin proximal promoter, interphotoreceptor retinoid-binding protein enhancer (RS/IRBPa), rhodopsin kinase (RK), liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific VGF gene promoter (Piccioli et al., Neuron, 15:373-84 (1995)), among others which will be apparent to the skilled artisan.

In some embodiments, a second promoter is a chicken beta-actin (CB) promoter. A chicken beta-actin promoter may be a short chicken beta-actin promoter or a long chicken beta-actin promoter. In some embodiments, a promoter (e.g., a chicken beta-actin promoter) comprises an enhancer sequence, for example a cytomegalovirus (CMV) enhancer sequence. A CMV enhancer sequence may be a short CMV enhancer sequence or a long CMV enhancer sequence. In some embodiments, a promoter comprises a long CMV enhancer sequence and a long chicken beta-actin promoter. In some embodiments, a promoter comprises a short CMV enhancer sequence and a short chicken beta-actin promoter. However, the skilled artisan recognizes that a short CMV enhancer may be used with a long CB promoter, and a long CMV enhancer may be used with a short CB promoter (and vice versa).

Aspects of the disclosure relate to expression constructs encoding a fusion protein that comprises a rapamycin-binding protein (FKBP). A rapamycin-binding protein (FKBP), as used herein, refers to a cytosolic receptor for rapamycin or rapalog and is highly conserved across species lines. Generally, FKBPs are proteins or protein domains which are capable of binding to rapamycin or to a rapalog and further forming a tripartite complex with an FRB-containing protein. Information concerning the nucleotide sequences, cloning, and other aspects of various FKBP species is known in the art, see e.g., Staendart et al, 1990, Nature 346, 671-674 (human FKBP12); Kay, 1996, Biochem. J. 314, 361-385 (review). Homologous FKBP proteins in other mammalian species, in yeast, and in other organisms are also known in the art and may be used in the fusion proteins disclosed herein. See e.g. Kay, 1996, Biochem. J. 314, 361-385 (review). An FKBP peptide is capable of binding to rapamycin or a rapalog and participating in a tripartite complex with a FRB-containing protein. Non-limiting examples of FKBR include naturally occurring peptide sequence derived from the human FKBP12 protein, a peptide sequence derived from another human FKBP, from a murine or other mammalian FKBP, or from some other animal, yeast or fungal FKBP. In some embodiments, the FKBP is FKBP12.

Aspects of the disclosure relate to expression constructs encoding a fusion protein that comprises a FKBP-rapamycin binding (FRB) domain. An FKBP-rapamycin binding (FRB) domain, as used herein, refers to polypeptide which are capable of forming a tripartite complex with an rapamycin-binding protein (FKBP) and rapamycin (or a rapalog). FRB domains are present in a number of naturally occurring proteins. Non-limiting examples of an FRB domain include FRAP proteins from human and other species, yeast proteins including Tor1 and Tor2, or a Candida FRAP homolog. Information concerning the nucleotide sequences, cloning, and other aspects of these proteins is already known in the art. For example, protein source reference/sequence accession numbers human FRAP Brown et al, 1994, Nature 369, 756-758; GenBank accession #L34075, NCBI Seq ID 508481; Chiu et al, 1994, PNAS USA 91, 12574 12578; Chen et al, 1995, PNAS USA 92, 4947 4951 murine RAPT1 Chiu et al, supra. yeast Tor1 Helliwell et al, 1994, Mol Cell Biol 5, 105-118; EMBL Accession #X74857 NCBI Se Id #468738 yeast Tor2 Kunz et al, 1993, Cell 73, 585-596; EMBL Accession #X71416, NCBI Seq ID 298027 Candida W095/33052 homolog. In some embodiments, the FRB is FRAP.

In some embodiments, a fusion protein comprises a FKBP or FRB and a DNA binding domain (DBD). A DNA binding domain, as used herein, refers to proteins domains that have DNA-binding capacity and thus have a specific or general affinity for single- or double-stranded DNA. Non-limiting examples of DNA binding domain include transcription factors, polymerases, histones, nucleases (e.g., zinc finger nucleases (ZFNs), transcription activator-like effector nucleases (TALENs), meganucleases (e.g., engineered meganucleases) and CRISPR-associated proteins (Cas nucleases)). In some embodiments, the DNA binding domain binds to the regulatable promoter of the first expression cassette of the isolated nucleic acid as described herein.

In some embodiments, a DNA binding protein is a zinc finger domain. A zinc finger domain, as used herein, refers to small protein motifs that contain multiple finger-like protrusions that make tandem contacts with their target molecule. Zinc finger domains are capable of binding bind DNA, RNA, protein, and/or lipid substrates. The binding of a zinc-finger domain to its target site juxtaposes three base pairs on DNA to a few amino acids in the α-helix structure. The identity of the amino acids at the contact site defines the DNA sequence recognition specificity of zinc fingers. Thus, by changing these amino acids, a high degree of selectivity can be achieved toward a given three base-pair DNA sequence (see, e.g., Cassandri, Zinc-finger proteins in health and disease, Cell Death Discovery volume 3, Article number: 17071 (2017)). In some embodiments, the zinc finger protein binds to DNA sequences. In some embodiments, the zinc finger domain binds to the regulatable promoter of the first expression cassette of the isolated nucleic acid as described herein. In some embodiments, the zinc finger domain is designed based on the regulatable promoter of the isolated nucleic acid.

In some embodiments, a DNA binding domain is a CRISPR-associated protein. The term “CRISPR” refers to “clustered regularly interspaced short palindromic repeats”, which are DNA loci containing short repetitions of base sequences. CRISPR loci form a portion of a prokaryotic adaptive immune system that confers resistance to foreign genetic material. Each CRISPR loci is flanked by short segments of “spacer DNA”, which are derived from viral genomic material. In the Type II CRISPR system, spacer DNA hybridizes to transactivating RNA (tracrRNA) and is processed into CRISPR-RNA (crRNA) and subsequently associates with CRISPR-associated proteins (Cas proteins) to form complexes that recognize and degrade foreign DNA. Non-limiting Examples of Cas proteins include, but are not limited to Cas9, dCas9, Cas6, Cas13, CasRX, Cpf1, and variants thereof. In some embodiments, the Cas protein is a dCas protein. A dCas protein, as used herein, refers to a Cas protein is modified (e.g. genetically engineered) to lack nuclease activity. For example, “dead” Cas9 (dCas9) protein binds to a target locus but does not cleave said locus. Non-limiting examples for dCas protein include dCas9, and dCas13.

In some aspects, the disclosure relates to expression constructs encoding one or more transcription transactivator domains. A transcription activator domain, as used herein, refers to a protein domain (transcription factor) that increases gene transcription of a gene or set of genes. Most activators are DNA-binding proteins that bind to enhancers or promoter-proximal elements. Non-limiting examples of transcriptional activator domains include p65, VP8, VP16, or VP64. In some embodiments, the transcription activator is a p65 domain.

In some embodiments, the transgene in the second expression cassettes encodes fusion proteins. A fusion protein, as used herein, refer to proteins comprises various component domains or sequences that are mutually heterologous in the sense that they do not occur together in the same arrangement in nature. More specifically, the component portions are not found in the same continuous polypeptide or nucleotide sequence or molecule in nature, at least not in the same order or orientation or with the same spacing present in the fusion protein as described herein.

In some embodiments, fusion proteins described herein contain at least one “receptor” or “ligand binding” domain comprising peptide sequence derived from a FKBP protein and at least one other domain, heterologous with respect to the receptor domain, but capable, upon oligomerization of the chimeric protein molecules, of triggering a cellular or biological response (e.g., binding to the regulatable promoter of the first expression cassette of the isolated nucleic acid described herein). In some embodiments, the other domains comprise a DNA-binding domain (e.g., zinc finger domain) and a transcription activation domain (e.g., p65 domain), paired such that oligomerization of the fusion proteins represents assembly of a transcription factor complex which triggers transcription of a gene (e.g., the first transgene of the isolated nucleic acid) linked to a DNA sequence recognized by (capable of specific binding interaction with) the DNA binding domain (e.g., the regulatable promoter of the first expression cassette of the isolated nucleic acid).

In some embodiments, a FRB domain is fused to a transcription activator domain. In some embodiments, a FRB domain is fused to a DNA binding domain. In some embodiments, a FKBP is fused to a DNA binding domain. In some embodiments, a FRB domain is fused to a DNA binding domain, and a FKBP is fused to a transcription activator domain. In some embodiments, a FKBP is fused to a transcription activator domain. In some embodiments, the FRB domain is fused to a transcription activator domain, and the FKBP is fused to a DNA binding domain. In some embodiments, a FRAP is fused to p65, and a FKBP12 is fused to a zinc finger domain.

In some embodiments, domains of a fusion protein are connected without a linker. In some embodiments, the domains of a fusion protein are connected via a linker. Suitable linkers are known in the art. (See, e.g., Chen et al., Fusion protein linkers: property, design and functionality, Adv Drug Deliv Rev. 2013 October; 65(10):1357-69). In some embodiments, an FKBP-DNA binding domain fusion protein comprises an amino acid sequence at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acid sequence of SEQ ID NO: 1. In some embodiments, the FRB-transcription activator fusion protein comprises an amino acid sequence at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acid sequence of SEQ ID NO: 2.

An exemplary amino acid sequence for FKBP12-zinc finger domain fusion protein is set forth in SEQ ID NO: 1.

MDYPAAKRVKLDSRERPYACPVESCDRRFSRSDELTRHIRIHTGQKPFQ
CRICMRNFSRSDHLTTHIRTHTGGGRRRKKRTSIETNIRVALEKSFLEN
QKPTSEEITMIADQLNMEKEVIRVWFCNRRQKEKRINTRGVQVETISPG
DGRTFPKRGQTCVVHYTGMLEDGKKFDSSRDRNKPFKFMLGKQEVIRGW
EEGVAQMSVGQRAKLTISPDYAYGATGHPGIIPPHATLVFDVELLKLEV
EGVQVETISPGDGRTFPKRGQTCVVHYTGMLEDGKKFDSSRDRNKPFKF
MLGKQEVIRGWEEGVAQMSVGQRAKLTISPDYAYGATGHPGIIPPHATL
VFDVELLKLETRGVQVETISPGDGRTFPKRGQTCVVHYTGMLEDGKKFD
SSRDRNKPFKFMLGKQEVIRGWEEGVAQMSVGQRAKLTISPDYAYGATG
HPGIIPPHATLVFDVELLKLETSY

An exemplary amino acid sequence for FRAP-p65 fusion protein is set forth in SEQ ID NO: 2.

MASRILWHEMWHEGLEEASRLYFGERNVKGMFEVLEPLHAMMERGPQTL
KETSFNQAYGRDLMEAQEWCRKYMKSGNVKDLLQAWDLYYHVFRRISKT
RDEFPTMVFPSGQISQASALAPAPPQVLPQAPAPAPAPAMVSALAQAPA
PVPVLAPGPPQAVAPPAPKPTQAGEGTLSEALLQLQFDDEDLGALLGNS
TDPAVFTDLASVDNSEFQQLLNQGIPVAPHTTEPMLMEYPEAITRLVTG
AQRPPDPAPAPLGAPGLPNGLLSGDEDFSSIADMDFSALLSQISSTSY

In some embodiments, a second transgene encode a FKBP-DNA binding domain fusion protein (e.g., FKBP12-zinc finger protein fusion protein) and a FRB-transcription activator fusion protein (e.g., FRAP-p65 fusion protein). In some embodiments, the second expression cassette is a multicistronic expression cassette.

A multicistronic expression cassette, as used herein, refers to expression cassettes that simultaneously express two or more separate proteins from the same mRNA. In some embodiments, the second expression cassette further comprises an IRES or a 2A self-cleaving peptide coding sequence. In some embodiments, the internal ribosome entry site (IRES) or the 2A peptide coding sequence is located between the FKBP-DNA binding domain fusion protein (e.g., FKBP12-zinc finger protein fusion protein) and a FRB-transcription activator fusion protein (e.g., FRAP-p65 fusion protein). An internal ribosome entry site (IRES), is an RNA element that allows for translation initiation in a cap-independent manner, as part of the greater process of protein synthesis. A 2A self-cleaving peptide, as used herein, refers to a class of 18-22 aa-long peptides, which can induce the cleaving of the recombinant protein in cell. In some embodiments, the second transgene comprises an IRES located between the FKBP-DNA binding domain fusion protein (e.g., FKBP12-zinc finger protein fusion protein) and a FRB-transcription activator fusion protein (e.g., FRAP-p65 fusion protein).

An exemplary nucleic acid sequence encoding FKBP12-zinc finger protein fusion protein-IRES-FRAP-p65 fusion protein is set forth in SEQ ID NO: 4. In some embodiments, the second transgene comprises a nucleic acid sequence at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to nucleic acid sequence of SEQ ID NO: 4.

ATGGCTTCTAGAATCCTCTGGCATGAGATGTGGCATGAAGGCCTGGAAGAGGCATCTCGTTT
GTACTTTGGGGAAAGGAACGTGAAAGGCATGTTTGAGGTGCTGGAGCCCTTGCATGCTATGA
TGGAACGGGGCCCCCAGACTCTGAAGGAAACATCCTTTAATCAGGCCTATGGTCGAGATTTA
ATGGAGGCCCAAGAGTGGTGCAGGAAGTACATGAAATCAGGGAATGTCAAGGACCTCCTCCA
AGCCTGGGACCTCTATTATCATGTGTTCCGACGAATCTCAAAGACTAGAGATGAGTTTCCCA
CCATGGTGTTTCCTTCTGGGCAGATCAGCCAGGCCTCGGCCTTGGCCCCGGCCCCTCCCCAA
GTCCTGCCCCAGGCTCCAGCCCCTGCCCCTGCTCCAGCCATGGTATCAGCTCTGGCCCAGGC
CCCAGCCCCTGTCCCAGTCCTAGCCCCAGGCCCTCCTCAGGCTGTGGCCCCACCTGCCCCCA
AGCCCACCCAGGCTGGGGAAGGAACGCTGTCAGAGGCCCTGCTGCAGCTGCAGTTTGATGAT
GAAGACCTGGGGGCCTTGCTTGGCAACAGCACAGACCCAGCTGTGTTCACAGACCTGGCATC
CGTCGACAACTCCGAGTTTCAGCAGCTGCTGAACCAGGGCATACCTGTGGCCCCCCACACAA
CTGAGCCCATGCTGATGGAGTACCCTGAGGCTATAACTCGCCTAGTGACAGGGGCCCAGAGG
CCCCCCGACCCAGCTCCTGCTCCACTGGGGGCCCCGGGGCTCCCCAATGGCCTCCTTTCAGG
AGATGAAGACTTCTCCTCCATTGCGGACATGGACTTCTCAGCCCTGCTGAGTCAGATCAGCT
CCACTAGTTATTAAGAATTCACGCGTCGAGCATGCATCTAGGGCGGCCAATTCCGCCCCTCT
CCCCCCCCCCCCTCTCCCTCCCCCCCCCCTAACGTTACTGGCCGAAGCCGCTTGGAATAAGG
CCGGTGTGCGTTTGTCTATATGTTATTTTCCACCATATTGCCGTCTTTTGGCAATGTGAGGG
CCCGGAAACCTGGCCCTGTCTTCTTGACGAGCATTCCTAGGGGTCTTTCCCCTCTCGCCAAA
GGAATGCAAGGTCTGTTGAATGTCGTGAAGGAAGCAGTTCCTCTGGAAGCTTCTTGAAGACA
AACAACGTCTGTAGCGACCCTTTGCAGGCAGCGGAACCCCCCACCTGGCGACAGGTGCCTCT
GCGGCCAAAAGCCACGTGTATAAGATACACCTGCAAAGGCGGCACAACCCCAGTGCCACGTT
GTGAGTTGGATAGTTGTGGAAAGAGTCAAATGGCTCTCCTCAAGCGTATTCAACAAGGGGCT
GAAGGATGCCCAGAAGGTACCCCATTGTATGGGATCTGATCTGGGGCCTCGGTGCACATGCT
TTACATGTGTTTAGTCGAGGTTAAAAAAACGTCTAGGCCCCCCGAACCACGGGGACGTGGTT
TTCCTTTGAAAAACACGATGATAAGCTTGCCACAACCCGGGATCCTCTAGAGTCGACATGGA
CTATCCTGCTGCCAAGAGGGTCAAGTTGGACTCTAGAGAACGCCCATATGCTTGCCCTGTCG
AGTCCTGCGATCGCCGCTTTTCTCGCTCGGATGAGCTTACCCGCCATATCCGCATCCACACA
GGCCAGAAGCCCTTCCAGTGTCGAATCTGCATGCGTAACTTCAGTCGTAGTGACCACCTTAC
CACCCACATCCGCACCCACACAGGCGGCGGCCGCAGGAGGAAGAAACGCACCAGCATAGAGA
CCAACATCCGTGTGGCCTTAGAGAAGAGTTTCTTGGAGAATCAAAAGCCTACCTCGGAAGAG
ATCACTATGATTGCTGATCAGCTCAATATGGAAAAAGAGGTGATTCGTGTTTGGTTCTGTAA
CCGCCGCCAGAAAGAAAAAAGAATCAACACTAGAGGAGTGCAGGTGGAAACCATCTCCCCGG
GAGACGGGCGCACCTTCCCCAAGCGCGGCCAGACCTGCGTGGTGCACTACACCGGGATGCTT
GAAGATGGAAAGAAATTTGATTCCTCCCGGGACAGAAACAAGCCCTTTAAGTTTATGCTAGG
CAAGCAGGAGGTGATCCGAGGCTGGGAAGAAGGGGTTGCCCAGATGAGTGTGGGTCAGAGAG
CCAAACTGACTATATCTCCAGATTATGCCTATGGTGCCACTGGGCACCCAGGCATCATCCCA
CCACATGCCACTCTCGTCTTCGATGTGGAGCTTCTAAAACTGGAAGTCGAGGGCGTGCAGGT
GGAAACCATCTCCCCAGGAGACGGGCGCACCTTCCCCAAGCGCGGCCAGACCTGCGTGGTGC
ACTACACCGGGATGCTTGAAGATGGAAAGAAATTTGATTCCTCCCGGGACAGAAACAAGCCC
TTTAAGTTTATGCTAGGCAAGCAGGAGGTGATCCGAGGCTGGGAAGAAGGGGTTGCCCAGAT
GAGTGTGGGTCAGAGAGCCAAACTGACTATATCTCCAGATTATGCCTATGGTGCCACTGGGC
ACCCAGGCATCATCCCACCACATGCCACTCTCGTCTTCGATGTGGAGCTTCTAAAACTGGAA
ACTAGAGGAGTGCAGGTGGAAACCATCTCCCCAGGAGACGGGCGCACCTTCCCCAAGCGCGG
CCAGACCTGCGTGGTGCACTACACCGGGATGCTTGAAGATGGAAAGAAATTTGATTCCTCCC
GGGACAGAAACAAGCCCTTTAAGTTTATGCTAGGCAAGCAGGAGGTGATCCGAGGCTGGGAA
GAAGGGGTTGCCCAGATGAGTGTGGGTCAGAGAGCCAAACTGACTATATCTCCAGATTATGC
CTATGGTGCCACTGGGCACCCAGGCATCATCCCACCACATGCCACTCTCGTCTTCGATGTGG
AGCTTCTAAAACTGGAAACTAGTTATTAA

In some embodiments, the FKBP-DNA binding domain fusion protein (e.g., FKBP12-zinc finger protein fusion protein) and a FRB-transcription activator fusion protein (e.g., FRAP-p65 fusion protein) forms a heterodimer in response to rapamycin or rapalog. Rapamycin was initially discovered as an antifungal metabolite produced by Streptomyces hygroscopicus from a soil sample of Easter Island (also known as Rapa Nui). Subsequently, rapamycin was found to possess immunosuppressive and anti-proliferative properties in mammalian cells, spurring an interest in identifying the mode of action of rapamycin. Rapamycin was shown to be a potent inhibitor of S6K1 activation, a serine/threonine kinase activated by a variety of agonists (Chung et al., 1992; Kuo et al., 1992; Price et al., 1992) and an important mediator of PI3 kinase signaling. Concurrently, the target of rapamycin (TOR) was identified in yeast and animal cells (Laplante and Sabatini, 2012; Loewith and Hall, 2011). Rapamycin functions by binding with high affinity to FKBP, and then to the large PI3K homolog FRAP (RAFT, mTOR), thereby acting as a heterodimerizer to join the two proteins together. In some embodiments, a rapalogs is a compound other than rapamycin, preferably of molecular weight below 5 kD, more preferably below 2.5 kD, which is capable of binding with an FKBP fusion protein and of forming a complex with an FKBP fusion protein and an FRB fusion protein. Rapalogs are rapamycin analogs also capable of inhibiting mTOR signaling. Rapalogs have been previously described, e.g., Abdel-Magid et al., Rapalogs Potential as Practical Alternatives to Rapamycin, ACS Medicinal Chemistry Letters 2019 10 (6), 843-845. In some embodiments, the rapalog is Sirolimus, Temsirolimus, or Everolimus.

In some embodiments, a second transgene encodes a FKBP-DNA binding domain fusion protein (e.g., FKBP12-zinc finger protein fusion protein) and a FRB-transcription activator fusion protein (e.g., FRAP-p65 fusion protein). The zinc finger protein is capable of binding to the regulatable promoter of the first expression cassette of the isolated nucleic acid. In some embodiments, in the absence of rapamycin or rapalog, the FKBP-DNA binding domain fusion protein (e.g., FKBP12-zinc finger protein fusion protein) does not form a heterodimer with a FRB-transcription activator fusion protein (e.g., FRAP-p65 fusion protein), thus the regulatable promoter is not activated. In other embodiments, in the presence of rapamycin or rapalog, the FKBP-DNA binding domain fusion protein (e.g., FKBP12-zinc finger protein fusion protein) forms a heterodimer with a FRB-transcription activator fusion protein (e.g., FRAP-p65 fusion protein), thus the regulatable promoter is activated by the transcription activator, driving the expression of the first transgene.

In some embodiments, the isolated nucleic acid described herein is a multicistronic expression construct. In some embodiments, multicistronic expression constructs comprise expression cassettes that are positioned in different ways. For example, in some embodiments, a multicistronic expression construct is provided in which a first expression cassette (e.g., the first expression cassette described herein) is positioned adjacent to a second expression cassette (e.g., the second expression cassette described herein). In some embodiments, a multicistronic expression construct is provided in which a first expression cassette comprises an intron, and a second expression cassette is positioned within the intron of the first expression cassette. In some embodiments, the second expression cassette, positioned within an intron of the first expression cassette, comprises a promoter and a nucleic acid sequence encoding a gene product operatively linked to the promoter.

The term “orientation” as used herein in connection with expression cassettes, refers to the directional characteristic of a given cassette or structure. In some embodiments, an expression cassette harbors a promoter 5′ of the encoding nucleic acid sequence, and transcription of the encoding nucleic acid sequence runs from the 5′ terminus to the 3′ terminus of the sense strand, making it a directional cassette (e.g. 5′-promoter/(intron)/encoding sequence-3′). Since virtually all expression cassettes are directional in this sense, those of skill in the art can easily determine the orientation of a given expression cassette in relation to a second nucleic acid structure, for example, a second expression cassette, a viral genome, or, if the cassette is comprised in an AAV construct, in relation to an AAV ITR.

For example, if a given nucleic acid construct comprises two expression cassettes in the configuration 5′-promoter 1/encoding sequence 1-promoter2/encoding sequence 2-3′,
>>>>>>>>>>>>>>>>>>>> >>>>>>>>>>>>>>>>>>>>>>>>>>

the expression cassettes are in the same orientation, the arrows indicate the direction of transcription of each of the cassettes. For another example, if a given nucleic acid construct comprises a sense strand comprising two expression cassettes in the configuration 5′-promoter 1/encoding sequence 1-encoding sequence 2/promoter 2-3′,
>>>>>>>>>>>>>>>>>>>>>>> <<<<<<<<<<<<<<<<<<<<<

the expression cassettes are in opposite orientation to each other and, as indicated by the arrows, the direction of transcription of the expression cassettes, are opposed. In this example, the strand shown comprises the antisense strand of promoter 2 and encoding sequence 2.

For another example, if an expression cassette is comprised in an AAV construct, the cassette can either be in the same orientation as an AAV ITR, or in opposite orientation. AAV ITRs are directional. For example, the 3′ITR would be in the same orientation as the promoter1/encoding sequence 1 expression cassette of the examples above, but in opposite orientation to the 5′ITR, if both ITRs and the expression cassette would be on the same nucleic acid strand.

In some embodiments, a multicistronic expression construct is provided that allows efficient expression of a first encoding nucleic acid sequence driven by a first promoter and of a second encoding nucleic acid sequence driven by a second promoter without the use of transcriptional insulator elements. Various configurations of such multicistronic expression constructs are provided herein, for example, expression constructs harboring a first expression cassette comprising an intron and a second expression cassette positioned within the intron, in either the same or opposite orientation as the first cassette. Other configurations are described in more detail elsewhere herein.

In some embodiments, multicistronic expression constructs are provided allowing for efficient expression of two or more encoding nucleic acid sequences. In some embodiments, the multicistronic expression construct comprises two expression cassettes. In some embodiments, a first expression cassette of a multicistronic expression construct as provided herein comprises a first RNA polymerase II promoter and a second expression cassette comprises a second RNA polymerase II promoter. In some embodiments, a first expression cassette of a multicistronic expression construct as provided herein comprises an RNA polymerase II promoter and a second expression cassette comprises an RNA polymerase III promoter.

In some embodiments, the first and/or the second transgene comprises a 3′-untranslated region (3′-UTR). In some embodiments, the disclosure relates to isolated nucleic acids comprising a first and/or a second transgene each comprises one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in the 3′ untranslated region (3′-UTR) of a transgene, for example between the last codon of a nucleic acid sequence encoding a transgene (e.g., a first transgene and/or a second transgene) and a poly A sequence.

In some embodiments, a transgene comprises one or more (e.g., 1, 2, 3, 4, 5, or more) miRNA binding sites that de-target expression of the first transgene (e.g., cytokine, toxin or growth factors) and/or the second transgene (e.g., FRAP-zinc finger domain fusion protein and FRB-p65 fusion protein) from immune cells (e.g., antigen presenting cells (APCs), such as macrophages, dendrites, etc.). Incorporation of miRNA binding sites for immune-associated miRNAs may de-target transgene (e.g., the first transgene and/or the second transgene described herein) expression from antigen presenting cells and thus reduce or eliminate immune responses (cellular and/or humoral) produced in the subject against products of the transgene, for example as described in US 2018/0066279, the entire contents of which are incorporated herein by reference.

In some embodiments, the first and/the second transgene comprises one or more (e.g., 1, 2, 3, 4, 5, or more) miRNA binding sites that de-target expression of the first and/or second transgene from liver cells. For example, in some embodiments, a transgene comprises one or more miR-122 binding sites.

As used herein an “immune cell-associated miRNA” is a miRNA preferentially expressed in cells of the immune system, such as an antigen presenting cell (APC). In some embodiments, an immune cell-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune cell-associated miRNA is expressed is a B cell, T cell, Killer T cell, Helper T cell, γδ T cell, dendritic cell, macrophage, monocyte, vascular endothelial cell, or other immune cell. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, 0X40, PD-1 (Programmed death-1), RT6, TCR (T cell receptor), Thy-1 (CD90), and TSA-2 (Thymic shared Ag-2). In some embodiments, the immune cell-associated miRNA is selected from: miR-106, miR-125a, miR-125b, miR-126a, miR-142, miR-146a, miR-15, miR-150, miR-155, miR-16, miR-17, miR-18, miR-181 a, miR-19a, miR-19b, miR-20, miR-21a, miR-223, miR-24-3p, miR-29a, miR-29b, miR-29c, miR-302a-3p, miR-30b, miR-33-5p, miR-34a, miR-424, miR-652-3p, miR-652-5p, miR-9-3p, miR-9-5p, miR-92a, and miR-99b-5p. In some embodiments, a transgene described herein comprises one or more binding sites for miR-142.

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, a first and a second transgene described herein comprises a Kozak sequence. A Kozak sequence is a nucleic acid motif comprising a consensus sequence that is found in eukaryotic mRNA and plays a role in initiation of protein translation. In some embodiments, the Kozak sequence is positioned between the intron and the transgene encoding the first and second transgene described herein

An isolated nucleic acid described by the disclosure may encode a first and a transgene that further comprises a polyadenylation (poly A) sequence. In some embodiments, a first and a second transgene comprises a poly A sequence is a rabbit beta-globulin (RBG) poly A sequence,

In some embodiments, the isolated nucleic acid comprises inverted terminal repeats. The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, one or more transgene (e.g., the first and second transgene described herein) and its regulatory sequences, and 5′ and 3′ AAV inverted terminal repeats (ITRs). The one or more transgene may comprise a region encoding, for example, a first transgene product (e.g., inhibitory nucleic acid, cytokine, toxin, or growth factors) and a second transgene (e.g., FRAP-zinc finger domain fusion protein and FRB-p65 fusion protein) and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.)

comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are selected from the group consisting of AAV1 ITR, AAV2 ITR, AAV3 ITR, AAV4 ITR, AAV5 ITR, and AAV6 ITR.

In some embodiments, the isolated nucleic acid as described herein comprises a 5′ AAV ITR, a first expression cassette described herein, a second expression cassette described herein, and a 3′ AAV ITR.

Also within the scope of the present disclosure are plasmids comprising the isolated nucleic acid described herein. An exemplary full plasmid sequence comprising an AAV vector comprising the first and the second expression cassettes is set forth in SEQ ID NO: 3. In some embodiments, the plasmid comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the nucleic acid sequence as set forth in SEQ ID NO: 3:

(SEQ ID NO: 3)
GATCTGCTAATGATGGGCGCTCGAGTAATGATGGGCGGTCGACTAATGATGGGCGCTCGAGT
AATGATGGGCGTCTAGCTAATGATGGGCGCTCGAGTAATGATGGGCGGTCGACTAATGATGG
GCGCTCGAGTAATGATGGGCGTCTAGCTAATGATGGGCGCTCGAGTAATGATGGGCGGTCGA
CTAATGATGGGCGCTCGAGTAATGATGGGCGTCTATTAATTCAACATTTTGACACCCCCATA
ATATTTTTCCAGAATTAACAGTATAAATTGCATCTCTTGTTCAAGAGTTCCCTATCACTCTC
TTTAATCACTACTCACAGTAACCTCAACTCCTGCCACAAGCTTGAATTTCCTGGAGGCTTGC
TGAAGGCTGTATGCTGAATGTAAGCTGGCAGACCTTCGTTTTGGCCACTGACTGACGAAGGT
CTCAGCTTACATTCAGGACACAAGGCCTGTTACTAGCACTCACATGGAACAAATGGCGCAAG
CGGCCGCGCCTCGACTGTGCCTTCTAGTTGCCAGCCATCTGTTGTTTGCCCCTCCCCCGTGC
CTTCCTTGACCCTGGAAGGTGCCACTCCCACTGTCCTTTCCTAATAAAATGAGGAAATTGCA
TCGCATTGTCTGAGTAGGTGTCATTCTATTCTTACCTCTGGTCGTTACATAACTTACGGTAA
ATGGCCCGCCTGGCTGACCGCCCAACGACCCCCGCCCATTGACGTCAATAATGACGTATGTT
CCCATAGTAACGCCAATAGGGACTTTCCATTGACGTCAATGGGTGGAGTATTTACGGTAAAC
TGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGG
CAGTACATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGCGGGGGGGGGGGG
GGGGCGCGCGCCAGGCGGGGCGGGGCGGGGCGAGGGGCGGGGCGGGGCGAGGCGGAGAGGTG
CGGCGGCAGCCAATCAGAGCGGCGCGCTCCGAAAGTTTCCTTTTATGGCGAGGCGGCGGCGG
CGGCGGCCCTATAAAAAGCGAAGCGCGCGGCGGGCGGGAGCGGGATCAGCCACCGCGGTGGC
GGCCTAGAGTCGACGAGGAACTGAAAAACCAGAAAGTTAACTGGTAAGTTTAGTCTTTTTGT
CTTTTATTTCAGGTCCCGGATCCGGTGGTGGTGCAAATCAAAGAACTGCTCCTCAGTGGATG
TTGCCTTTACTTCTAGGCCTGTACGATGGCTTCTAGAATCCTCTGGCATGAGATGTGGCATG
AAGGCCTGGAAGAGGCATCTCGTTTGTACTTTGGGGAAAGGAACGTGAAAGGCATGTTTGAG
GTGCTGGAGCCCTTGCATGCTATGATGGAACGGGGCCCCCAGACTCTGAAGGAAACATCCTT
TAATCAGGCCTATGGTCGAGATTTAATGGAGGCCCAAGAGTGGTGCAGGAAGTACATGAAAT
CAGGGAATGTCAAGGACCTCCTCCAAGCCTGGGACCTCTATTATCATGTGTTCCGACGAATC
TCAAAGACTAGAGATGAGTTTCCCACCATGGTGTTTCCTTCTGGGCAGATCAGCCAGGCCTC
GGCCTTGGCCCCGGCCCCTCCCCAAGTCCTGCCCCAGGCTCCAGCCCCTGCCCCTGCTCCAG
CCATGGTATCAGCTCTGGCCCAGGCCCCAGCCCCTGTCCCAGTCCTAGCCCCAGGCCCTCCT
CAGGCTGTGGCCCCACCTGCCCCCAAGCCCACCCAGGCTGGGGAAGGAACGCTGTCAGAGGC
CCTGCTGCAGCTGCAGTTTGATGATGAAGACCTGGGGGCCTTGCTTGGCAACAGCACAGACC
CAGCTGTGTTCACAGACCTGGCATCCGTCGACAACTCCGAGTTTCAGCAGCTGCTGAACCAG
GGCATACCTGTGGCCCCCCACACAACTGAGCCCATGCTGATGGAGTACCCTGAGGCTATAAC
TCGCCTAGTGACAGGGGCCCAGAGGCCCCCCGACCCAGCTCCTGCTCCACTGGGGGCCCCGG
GGCTCCCCAATGGCCTCCTTTCAGGAGATGAAGACTTCTCCTCCATTGCGGACATGGACTTC
TCAGCCCTGCTGAGTCAGATCAGCTCCACTAGTTATTAAGAATTCACGCGTCGAGCATGCAT
CTAGGGCGGCCAATTCCGCCCCTCTCCCCCCCCCCCCTCTCCCTCCCCCCCCCCTAACGTTA
CTGGCCGAAGCCGCTTGGAATAAGGCCGGTGTGCGTTTGTCTATATGTTATTTTCCACCATA
TTGCCGTCTTTTGGCAATGTGAGGGCCCGGAAACCTGGCCCTGTCTTCTTGACGAGCATTCC
TAGGGGTCTTTCCCCTCTCGCCAAAGGAATGCAAGGTCTGTTGAATGTCGTGAAGGAAGCAG
TTCCTCTGGAAGCTTCTTGAAGACAAACAACGTCTGTAGCGACCCTTTGCAGGCAGCGGAAC
CCCCCACCTGGCGACAGGTGCCTCTGCGGCCAAAAGCCACGTGTATAAGATACACCTGCAAA
GGCGGCACAACCCCAGTGCCACGTTGTGAGTTGGATAGTTGTGGAAAGAGTCAAATGGCTCT
CCTCAAGCGTATTCAACAAGGGGCTGAAGGATGCCCAGAAGGTACCCCATTGTATGGGATCT
GATCTGGGGCCTCGGTGCACATGCTTTACATGTGTTTAGTCGAGGTTAAAAAAACGTCTAGG
CCCCCCGAACCACGGGGACGTGGTTTTCCTTTGAAAAACACGATGATAAGCTTGCCACAACC
CGGGATCCTCTAGAGTCGACATGGACTATCCTGCTGCCAAGAGGGTCAAGTTGGACTCTAGA
GAACGCCCATATGCTTGCCCTGTCGAGTCCTGCGATCGCCGCTTTTCTCGCTCGGATGAGCT
TACCCGCCATATCCGCATCCACACAGGCCAGAAGCCCTTCCAGTGTCGAATCTGCATGCGTA
ACTTCAGTCGTAGTGACCACCTTACCACCCACATCCGCACCCACACAGGCGGCGGCCGCAGG
AGGAAGAAACGCACCAGCATAGAGACCAACATCCGTGTGGCCTTAGAGAAGAGTTTCTTGGA
GAATCAAAAGCCTACCTCGGAAGAGATCACTATGATTGCTGATCAGCTCAATATGGAAAAAG
AGGTGATTCGTGTTTGGTTCTGTAACCGCCGCCAGAAAGAAAAAAGAATCAACACTAGAGGA
GTGCAGGTGGAAACCATCTCCCCGGGAGACGGGCGCACCTTCCCCAAGCGCGGCCAGACCTG
CGTGGTGCACTACACCGGGATGCTTGAAGATGGAAAGAAATTTGATTCCTCCCGGGACAGAA
ACAAGCCCTTTAAGTTTATGCTAGGCAAGCAGGAGGTGATCCGAGGCTGGGAAGAAGGGGTT
GCCCAGATGAGTGTGGGTCAGAGAGCCAAACTGACTATATCTCCAGATTATGCCTATGGTGC
CACTGGGCACCCAGGCATCATCCCACCACATGCCACTCTCGTCTTCGATGTGGAGCTTCTAA
AACTGGAAGTCGAGGGCGTGCAGGTGGAAACCATCTCCCCAGGAGACGGGCGCACCTTCCCC
AAGCGCGGCCAGACCTGCGTGGTGCACTACACCGGGATGCTTGAAGATGGAAAGAAATTTGA
TTCCTCCCGGGACAGAAACAAGCCCTTTAAGTTTATGCTAGGCAAGCAGGAGGTGATCCGAG
GCTGGGAAGAAGGGGTTGCCCAGATGAGTGTGGGTCAGAGAGCCAAACTGACTATATCTCCA
GATTATGCCTATGGTGCCACTGGGCACCCAGGCATCATCCCACCACATGCCACTCTCGTCTT
CGATGTGGAGCTTCTAAAACTGGAAACTAGAGGAGTGCAGGTGGAAACCATCTCCCCAGGAG
ACGGGCGCACCTTCCCCAAGCGCGGCCAGACCTGCGTGGTGCACTACACCGGGATGCTTGAA
GATGGAAAGAAATTTGATTCCTCCCGGGACAGAAACAAGCCCTTTAAGTTTATGCTAGGCAA
GCAGGAGGTGATCCGAGGCTGGGAAGAAGGGGTTGCCCAGATGAGTGTGGGTCAGAGAGCCA
AACTGACTATATCTCCAGATTATGCCTATGGTGCCACTGGGCACCCAGGCATCATCCCACCA
CATGCCACTCTCGTCTTCGATGTGGAGCTTCTAAAACTGGAAACTAGTTATTAAGGCCAGAC
ATGATAAGATACATTGATGAGTTTGGACAAACCACAACTAGAATGCAGTGAAAAAAATGCTT
TATTTGTGAAATTTGTGATGCTATTGCTTTATTTGTAACCATTATAAGCTGCAATAAACAAG
TTAACAACAACAATTGCATTCATTTTATGTTTCAGGTTCAGGGGGAGATGTGGGAGGTTTTT
TAAAGCAAGTAAAACCTCTACAAATGTGGTAATCGATAAGGATCTAGGAACCCCTAGTGATG
GAGTTGGCCACTCCCTCTCTGCGCGCTCGCTCGCTCACTGAGGCCGCCCGGGCAAAGCCCGG
GCGTCGGGCGACCTTTGGTCGCCCGGCCTCAGTGAGCGAGCGAGCGCGCAGAGAGGGAGTGG
CCAACCCCCCCCCCCCCCCCCCTGCAGCCTGGCGTAATAGCGAAGAGGCCCGCACCGATCGC
CCTTCCCAACAGTTGCGTAGCCTGAATGGCGAATGGCGCGACGCGCCCTGTAGCGGCGCATT
AAGCGCGGCGGGTGTGGTGGTTACGCGCAGCGTGACCGCTACACTTGCCAGCGCCCTAGCGC
CCGCTCCTTTCGCTTTCTTCCCTTCCTTTCTCGCCACGTTCGCCGGCTTTCCCCGTCAAGCT
CTAAATCGGGGGCTCCCTTTAGGGTTCCGATTTAGTGCTTTACGGCACCTCGACCCCAAAAA
ACTTGATTAGGGTGATGGTTCACGTAGTGGGCCATCGCCCTGATAGACGGTTTTTCGCCCTT
TGACGTTGGAGTCCACGTTCTTTAATAGTGGACTCTTGTTCCAAACTGGAACAACACTCAAC
CCTATCTCGGTCTATTCTTTTGATTTATAAGGGATTTTGCCGATTTCGGCCTATTGGTTAAA
AAATGAGCTGATTTAACAAAAATTTAACGCGAATTTTAACAAAATATTAACGTTTACAATTT
CCTGATGCGGTATTTTCTCCTTACGCATCTGTGCGGTATTTCACACCGCATATGGTGCACTC
TCAGTACAATCTGCTCTGATGCCGCATAGTTAAGCCAGCCCCGACACCCGCCAACACCCGCT
GACGCGCCCTGACGGGCTTGTCTGCTCCCGGCATCCGCTTACAGACAAGCTGTGACCGTCTC
CGGGAGCTGCATGTGTCAGAGGTTTTCACCGTCATCACCGAAACGCGCGAGACGAAAGGGCC
TCGTGATACGCCTATTTTTATAGGTTAATGTCATGATAATAATGGTTTCTTAGACGTCAGGT
GGCACTTTTCGGGGAAATGTGCGCGGAACCCCTATTTGTTTATTTTTCTAAATACATTCAAA
TATGTATCCGCTCATGAGACAATAACCCTGATAAATGCTTCAATAATATTGAAAAAGGAAGA
GTATGAGTATTCAACATTTCCGTGTCGCCCTTATTCCCTTTTTTGCGGCATTTTGCCTTCCT
GTTTTTGCTCACCCAGAAACGCTGGTGAAAGTAAAAGATGCTGAAGATCAGTTGGGTGCACG
AGTGGGTTACATCGAACTGGATCTCAACAGCGGTAAGATCCTTGAGAGTTTTCGCCCCGAAG
AACGTTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTTGAGTA
CTCACCAGTCACAGAAAAGCATCTTACGGATGGCATGACAGTAAGAGAATTATGCAGTGCTG
CCATAACCATGAGTGATAACACTGCGGCCAACTTACTTCTGACAACGATCGGAGGACCGAAG
GAGCTAACCGCTTTTTTGCACAACATGGGGGATCATGTAACTCGCCTTGATCGTTGGGAACC
GGAGCTGAATGAAGCCATACCAAACGACGAGCGTGACACCACGATGCCTGTAGCAATGGCAA
CAACGTTGCGCAAACTATTAACTGGCGAACTACTTACTCTAGCTTCCCGGCAACAATTAATA
GACTGGATGGAGGCGGATAAAGTTGCAGGACCACTTCTGCGCTCGGCCCTTCCGGCTGGCTG
GTTTATTGCTGATAAATCTGGAGCCGGTGAGCGTGGGTCTCGCGGTATCATTGCAGCACTGG
GGCCAGATGGTAAGCCCTCCCGTATCGTAGTTATCTACACGACGGGGAGTCAGGCAACTATG
GATGAACGAAATAGACAGATCGCTGAGATAGGTGCCTCACTGATTAAGCATTGGTAACTGTC
AGACCAAGTTTACTCATATATACTTTAGATTGATTTAAAACTTCATTTTTAATTTAAAAGGA
TCTAGGTGAAGATCCTTTTTGATAATCTCATGACCAAAATCCCTTAACGTGAGTTTTCGTTC
CACTGAGCGTCAGACCCCGTAGAAAAGATCAAAGGATCTTCTTGAGATCCTTTTTTTCTGCG
CGTAATCTGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACCAAATACT
GTCCTTCTAGTGTAGCCGTAGTTAGGCCACCACTTCAAGAACTCTGTAGCACCGCCTACATA
CCTCGCTCTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAGCGTGAGCA
TTGAGAAAGCGCCACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGGCCTTTTG
CTCACATGTTCTTTCCTGCGTTATCCCCTGATTCTGTGGATAACCGTATTACCGCCTTTGAG
TGAGCTGATACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTAATGCAGGG
CTGCAGGGGGGGGGGGGGGGGGGTTGGCCACTCCCTCTCTGCGCGCTCGCTCGCTCACTGAG
GCCGGGCGACCAAAGGTCGCCCGACGCCCGGGCTTTGCCCGGGCGGCCTCAGTGAGCGAGCG
AGCGCGCAGAGAGGGAGTGGCCAACTCCATCACTAGGGGTTCCTA

In some embodiments, the isolated nucleic acid comprises two flanking long terminal repeats (LTRs). The isolated nucleic acids of the disclosure may be recombinant adeno-associated virus (AAV) vectors (rAAV vectors).

In some embodiments, the isolated nucleic acid described herein can be packaged for delivery to a subject via a non-viral platform. In some embodiments, the isolated nucleic acid described herein can be delivered to a subject via closed-ended linear duplex DNA (ceDNA). Delivery of a transgene (e.g., first and second transgene described herein) via ceDNA has been described previously, see e.g., WO2017152149, the entire contents of which are incorporated herein by reference. In some embodiments, the nucleic acids having asymmetric terminal sequences (e.g., asymmetric interrupted self-complementary sequences) form closed-ended linear duplex DNA structures (e.g., ceDNA) that, in some embodiments, exhibit reduced immunogenicity compared to currently available gene delivery vectors. In some embodiments, ceDNA behaves as linear duplex DNA under native conditions and transforms into single-stranded circular DNA under denaturing conditions. Without wishing to be bound by any particular theory, ceDNA are useful, in some embodiments, for the delivery of a transgene (e.g., first and second transgene described herein) to a subject.

Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities (e.g., tissue tropism). Thus, an rAAV having a capsid appropriate for the tissue being targeted can be selected.

In some embodiments, the rAAV of the present disclosure comprises a capsid protein containing the isolated nucleic acid described herein.

Methods for obtaining recombinant AAVs having a desired capsid protein are well known in the art. (See, for example, US 2003/0138772, the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein has a tropism for ocular tissues or muscle tissue. In some embodiments, an AAV capsid protein targets ocular cell types (e.g., photoreceptor cells, retinal cells, etc.).

In some embodiments, an AAV capsid protein is of an AAV serotype selected from the group consisting of AAV1, AAV2, AAV3, AAV4, AAV5, AAV6, AAV7, AAV8, AAV9, AAV9.hr, AAVrh8, AAVrh10, AAVrh39, AAVrh43, AAV.PHP, and variants of any of the foregoing. In some embodiments, an AAV capsid protein is of a serotype derived from a non-human primate, for example AAVrh8 serotype. In some embodiments, the capsid protein is of AAV serotype 6 (e.g., AAV6 capsid protein), AAV serotype 8 (e.g., AAV8 capsid protein), AAV serotype 2 (e.g., AAV2 capsid protein), AAV serotype 5 (e.g., AAV5 capsid protein), AAV serotype 9 (e.g., AAV9 capsid protein), or AAVv66 serotype (e.g., AAVv66 capsid protein). In some embodiments, the AAV capsid protein with desired tissue tropism can be selected from AAV capsid proteins isolated from mammals (e.g., tissue from a subject). (See, for example, WO2010138263A2 and WO2018071831, the entire contents of which are incorporated herein by reference).

In some embodiments, the rAAV described herein is a single stranded AAV (ssAAV). An ssAAV, as used herein, refers to an rAAV with the coding sequence and complementary sequence of the transgene expression cassette on separate strands and are packaged in separate viral capsids.

The components to be cultured in the host cell to package an rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing an isolated nucleic acid that comprises (i) a first expression cassette comprising a regulatable promoter sequence operably linked to a first transgene; and (ii) a second expression cassette comprising second promoter operably linked to a second transgene, wherein the second transgene encodes a FKBP-rapamycin binding (FRB) domain, a transcription activator domain, a rapamycin-binding protein (FKBP), and a DNA binding domain that specifically binds to a portion of the regulatable promoter sequence of (i). In some embodiments, the host cell comprises the vector (e.g., AAV vector) or the ceDNA described herein. In some embodiments, the host further comprises a rapamycin or a rapalog. In some embodiments, the rapamycin or rapalog is present in the host cell at a concentration of between 5 and 3000 nM, or any intermediate number in between. In some embodiments, the rapamycin or rapalog is present in the host cell at a concentration of between 10 and 2000 nM, or any intermediate number in between. In some embodiments, the rapamycin or rapalog is present in the host cell at a concentration of 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1000 nM, 1500 nM, 2000 nM, 2500 nM, or 3000 nM.

. A “host cell” refers to any cell that harbors, or is capable of harboring, a substance of interest. Often a host cell is a mammalian cell. In some embodiments, a host cell is a photoreceptor cell, retinal pigment epithelial cell, keratinocyte, corneal cell, and/or a tumor cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a neuron, a photoreceptor cell, a pigmented retinal epithelial cell, or a glial cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, an anellovirus vector (e.g., Anellovirus vector as described in US20200188456A1), etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

### AAV-Mediated Delivery of Transgenes

Aspects of the instant disclosure relate to compositions comprising a recombinant AAV comprising a capsid protein and a nucleic acid encoding (i) a first expression cassette comprising a regulatable promoter sequence operably linked to a first transgene; and (ii) a second expression cassette comprising second promoter operably linked to a second transgene, wherein the second transgene encodes a FKBP-rapamycin binding (FRB) domain, a transcription activator domain, a rapamycin-binding protein (FKBP), and a DNA binding domain that specifically binds to a portion of the regulatable promoter sequence of (i). In some embodiments, the nucleic acid further comprises AAV ITRs.

The isolated nucleic acids, plasmids, rAAVs, and compositions comprising the isolated nucleic acid described herein, the plasmids described herein, or the rAAV described herein of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human. In some embodiments, the subject is a human.

Delivery of the rAAVs to a mammalian subject may be by, for example, intraocular injection, subretinal injection, topical administration, intramuscular injection or by administration into the bloodstream of the mammalian subject. Administration into the bloodstream may be by injection into a vein, an artery, or any other vascular conduit. Non-limiting exemplary methods of intramuscular administration of the rAAV include Intramuscular (IM) Injection and Intravascular Limb Infusion. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. In some embodiments, an rAAV or a composition (e.g., composition containing the isolated nucleic acid or the rAAV) as described in the disclosure is administered by intravitreal injection. In some embodiments, an rAAV or a composition (e.g., composition containing the isolated nucleic acid or the rAAV) as described in the disclosure is administered by intraocular injection. In some embodiments, an rAAV or a composition (e.g., composition containing the isolated nucleic acid or the rAAV) as described in the disclosure is administered by subretinal injection. In some embodiments, an rAAV or a composition (e.g., composition containing the isolated nucleic acid or the rAAV) as described in the disclosure is administered by intravenous injection. In some embodiments, an rAAV or a composition (e.g., composition containing the isolated nucleic acid or the rAAV) as described in the disclosure is administered by intramuscular injection. In some embodiments, an rAAV or a composition (e.g., composition containing the isolated nucleic acid or the rAAV) as described in the disclosure is administered by intratumoral injection.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

In some embodiments, a composition further comprises a pharmaceutically acceptable carrier. Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The rAAVs or the compositions (e.g., composition containing the isolated nucleic acid or the rAAV described herein) are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intravitreal delivery to the eye), intraocular injection, subretinal injection, oral, inhalation (including intranasal and intratracheal delivery), intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine an rAAV virion dose range to treat a patient having a particular disease or disorder based on the aforementioned factors, as well as other factors that are well known in the art.

An effective amount of rAAVs or composition (e.g., composition containing the isolated nucleic acid or the rAAV described herein) is an amount sufficient to target infect an animal, target a desired tissue (e.g., muscle tissue, ocular tissue, etc.). In some embodiments, an effective amount of an rAAV is administered to the subject during a pre-symptomatic stage of degenerative disease. In some embodiments, a subject is administered an rAAV or composition after exhibiting one or more signs or symptoms of degenerative disease. In some embodiments, the effective amount will depend primarily on factors such as the species, age, weight, health of the subject, and the tissue to be targeted, and may thus vary among animal and tissue. For example, an effective amount of the rAAV is generally in the range from about 1 ml to about 100 ml of solution containing from about 106 to 1016 genome copies (e.g., from 1×10⁶ to 1×10¹⁶, inclusive). In some embodiments, an effective amount of an rAAV ranges between 1×10⁹ and 1×10¹⁴ genome copies of the rAAV. In some cases, a dosage between about 10¹¹ to 10¹² rAAV genome copies is appropriate. In some embodiments, a dosage of between about 10¹¹ to 10¹³ rAAV genome copies is appropriate. In some embodiments, a dosage of between about 10¹¹ to 10¹⁴ rAAV genome copies is appropriate. In some embodiments, a dosage of between about 10¹¹ to 10¹⁵ rAAV genome copies is appropriate. In some embodiments, a dosage of about 10¹² to 10¹⁴ rAAV genome copies is appropriate. In some embodiments, a dosage of about 10¹³ to 10¹⁴ rAAV genome copies is appropriate. In some embodiments, a dosage of about 1×10¹², about 1.1×10¹², about 1.2×10¹², about 1.3×10¹², about 1.4×10¹², about 1.5×10¹², about 1.6×10¹², about 1.7×10¹², about 1.8×10¹², about 1.9×10¹², about 1×10¹³, about 1.1×10¹³, about 1.2×10¹³, about 1.3×10¹³, about 1.4×10¹³, about 1.5×10¹³, about 1.6×10¹³, about 1.7×10¹³, about 1.8×10¹³, about 1.9×10¹³, or about 2.0×10¹⁴ vector genome (vg) copies per kilogram (kg) of body weight is appropriate. In some embodiments, a dosage of between about 4×10¹² to 2×10¹³ rAAV genome copies is appropriate. In some embodiments a dosage of about 1.5×10¹³ vg/kg by intravenous administration is appropriate. In certain embodiments, 10¹²-10¹³ rAAV genome copies is effective to target tissues. In certain embodiments, 10¹³-10¹⁴ rAAV genome copies is effective to target tissues effective to target tissues (e.g., the eye).

In some embodiments, the rAAV described herein is administered to the subject once a day, once a week, once every two weeks, once a month, once every 2 months, once every 3 months, once every 6 months, once a year, or once in a lifetime of the subject.

An effective amount of rAAVs or composition (e.g., composition containing the isolated nucleic acid or the rAAV described herein) may also depend on the mode of administration. For example, targeting an ocular (e.g., corneal) tissue by intrastromal administration or subcutaneous injection may require different (e.g., higher or lower) doses, in some cases, than targeting an ocular (e.g., corneal) tissue by another method (e.g., systemic administration, topical administration). In some embodiments, intrastromal injection (IS) of rAAV having certain serotypes (e.g., AAV2, AAV5, AAV6, AAV6.2, AAV7, AAV8, AAV9, AAVrh.8, AAVrh.10, AAVrh.39, and AAVrh.43) mediates efficient transduction of ocular (e.g., corneal, retinal, etc.) cells. Thus, in some embodiments, the injection is intrastromal injection (IS). In some embodiments, the injection is topical administration (e.g., topical administration to an eye). In some cases, multiple doses of a rAAV are administered.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well-known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf-life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either intravitreally, intraocularly, subretinally, subcutaneously, intraopancreatically, intranasally, parenterally, intravenously, intramuscularly, intrathecally, orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that it is easily syringed. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the first transgene (e.g., inhibitor nucleic acid) and the second transgene (e.g., FRAP-zinc finger domain fusion protein and FRB-p65 fusion protein) described herein is delivered to the subject by ceDNA. Any compositions containing ceDNA described herein are also within the scope of the present disclosure. In some embodiments, the ceDNA and the compositions thereof can be administered to the subject using any suitable method described herein. In some embodiments, delivery of an effective amount of the ceDNA described herein by injection is in an amount such that it is sufficient to express an effective amount of an ceDNA encoding the first transgene (e.g., inhibitor nucleic acid) and the second transgene (e.g., FRAP-zinc finger domain fusion protein and FRB-p65 fusion protein). In some aspects, the disclosure relates to the recognition that one potential side-effect for administering an AAV to a subject is an immune response in the subject to the AAV, including inflammation. In some embodiments, a subject is immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, Solu-Medrol, Lansoprazole, trimethoprim/sulfamethoxazole, methotrexate, and any combination thereof. In some embodiments, the immunosuppression regimen comprises administering sirolimus, prednisolone, lansoprazole, trimethoprim/sulfamethoxazole, or any combination thereof.

In some embodiments, methods described by disclosure further comprise the step inducing immunosuppression (e.g., administering one or more immunosuppressive agents) in a subject prior to the subject being administered an rAAV (e.g., an rAAV or pharmaceutical composition as described by the disclosure). In some embodiments, a subject is immunosuppressed (e.g., immunosuppression is induced in the subject) between about 30 days and about 0 days (e.g., any time between 30 days until administration of the rAAV, inclusive) prior to administration of the rAAV to the subject. In some embodiments, the subject is pre-treated with immune suppression (e.g., rituximab, sirolimus, and/or prednisone) for at least 7 days.

In some embodiments, the methods described in this disclosure further comprise co-administration or prior administration of an agent to a subject administered an rAAV or pharmaceutical composition comprising an rAAV of the disclosure. In some embodiments, the agent is selected from a group consisting of Miglustat, Keppra, Prevacid, Clonazepam, and any combination thereof.

In some embodiments, immunosuppression of a subject maintained during and/or after administration of a rAAV or pharmaceutical composition. In some embodiments, a subject is immunosuppressed (e.g., administered one or more immunosuppressants) for between 1 day and 1 year after administration of the rAAV or pharmaceutical composition.

### Methods

Aspects of the disclosure relate to methods for treating a disease in a subject in need thereof, the method comprising: (i) administering to the subject a therapeutically effective amount of an isolated nucleic acid, vector, recombinant lentivirus, rAAV, ceDNA, host cell or pharmaceutical composition as described herein; and (ii) administering to the subject an effective amount of rapamycin or a rapalog.

Another aspects of the disclosure relates to A method for treating an alpha 1-antitrypsin associated disorder in a subject in need thereof, the method comprising: (i) administering to the subject a therapeutically effective amount of the isolated nucleic acid, the vector, the recombinant lentivirus, the rAAV, the ceDNA, the host cell or the pharmaceutical composition as described herein; and (ii) administering to the subject an effective amount of rapamycin or rapalog. In some embodiments, the rAAV encodes for an inhibitory nucleic acid targeting a mutant form of AAT. In some embodiments the inhibitory nucleic acid targeting the mutant form of AAT is a small RNA (e.g., miRNA, siRNA, shRNA, or AmiRNA). In some embodiments, the method further comprising supplementing the subject with wild-type AAT.

Another aspects of the disclosure relates to a method for treating Huntington's disease in a subject in need thereof, the method comprising: (i) administering to the subject a therapeutically effective amount of the isolated nucleic acid, the vector, the recombinant lentivirus, the rAAV, the ceDNA, the host cell or the pharmaceutical composition as described herein; and (ii) administering to the subject an effective amount of rapamycin or rapalog. In some embodiments, the rAAV encodes for an inhibitory nucleic acid targeting human huntingtin gene (HTT). In some embodiments the inhibitory nucleic acid targeting the mutant form of AAT is a small RNA (e.g., miRNA, siRNA, shRNA, or AmiRNA). In some embodiments, the small RNA targeting human huntingtin gene (HTT) gene is encoded by a nucleic acid comprising a sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleic acid sequence set forth in SEQ ID NO: 5.

In some embodiments, the subject is a human. In some embodiments, the subject is a non-human mammal. Non-limiting examples of non-human mammals are mouse, rat, cat, dog, sheep, rabbit, horse, cow, goat, pig, guinea pig, hamster, chicken, turkey, or a non-human primate.

Alleviating a disease (e.g., AAT or Huntington disease) includes delaying the development or progression of the disease, or reducing disease severity. Alleviating the disease does not necessarily require curative results. As used therein, “delaying” the development of a disease (e.g., AAT or Huntington disease) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

In some embodiments, the level of rapamycin or rapalog does not induce immunosuppression in the subject.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

As described herein, the first transgene is designed to express a molecule having an effect on a disease. It is within the scope of the present disclosure that any protein or inhibitory nucleic acid associated with any disease can be designed to be expressed by the first transgene. Non-limiting examples of diseases include Huntington's chorea, cystic fibrosis, alpha1 antitrypsin deficiency, muscular dystrophy, Hunter's syndrome, Lesch-Nyhan syndrome, Down's syndrome, Tay-Sachs disease, hemophilias, phenylketonuria, thalassemias, and sickle-cell anemia, cancer of the lung, breast, colon, prostate, kidney, pancreas, brain, bones, ovary, testes, and lymphatic organs.

In some embodiments, the isolated nucleic acid, the vector, the recombinant lentivirus, the rAAV, the ceDNA, the host cell or the pharmaceutical composition as described herein can be administered concurrently with the rapamycin or rapalog. In some embodiments, the isolated nucleic acid, the vector, the recombinant lentivirus, the rAAV, the ceDNA, the host cell or the pharmaceutical composition as described herein can be administered sequentially with the rapamycin or rapalog. In some embodiments, the isolated nucleic acid, the vector, the recombinant lentivirus, the rAAV, the ceDNA, the host cell or the pharmaceutical composition as described herein can be administered at different frequencies with the administration of rapamycin or rapalog.

Another aspects of the disclosure also provides method for modulating transgene expression in a subject, the method comprising: (i) administering to the subject a therapeutically effective amount of the isolated nucleic acid, the vector, the recombinant lentivirus, the rAAV, the ceDNA, the host cell or the pharmaceutical composition as described herein; (ii) measuring expression of transgene in the subject; (iii) administering more or less rapalog based on expression level measured in (ii). The expression of the first transgene may be modulated by the concentration of rapamycin or rapalog. In some embodiments, if the level of first transgene is lower than a control expression level in the subject. A control expression level of the first transgene is determined by the pharmacodynamics and pharmacokinetics of the first transgene product, and is known in the art. In some embodiments, if the level of the first transgene produce is higher than the control expression level, the amount of rapamycin or rapalog can be decreased. In some embodiments, if the level of the first transgene produce is lower than the control expression level, the amount of rapamycin or rapalog can be increased. The level of the transgene in the subject can be measured using routine methods in the art, such as RNA solution hybridization, nuclease protection, Northern hybridization, reverse transcription, gene expression monitoring with a microarray, antibody binding, enzyme linked immunosorbent assay (ELISA), nucleic acid sequencing, Western blotting, radioimmunoassay (RIA), other immunoassays, fluorescence activated cell analysis (FACS), or any other technique or combination of techniques that can detect the level of the first transgene (e.g., in a subject or a sample obtained from a subject). In some embodiments, a higher level of the first transgene is, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more higher than a desired level of the first transgene. In some embodiments, a lower level of the first transgene is, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more lower than a desired level of the first transgene.

In some embodiments, the amount of rapamycin or rapalog administered to the subject can be adjusted/modulated according to the level of the first transgene in the subject. In some embodiments, the level of rapamycin or rapalog does not induce immunosuppression in the subject. In some embodiments, when the expression level of the first transgene is higher relative to the control level, the concentration of rapalog administered to the subject can be the same or decreased. In some embodiments, the concentration of rapalog can be decreased at least 10%, 15%, 20%, 25%, 30%, 35%, 40%, 45%, 50% or more. In some embodiments, when the expression level of the first transgene is lower relative to the control level, the concentration of rapalog administered to the subject can be increased. In some embodiments, the concentration of rapalog can be increase at least 10%, 15%, 20%, 25%, 30%, 35%, 40%, 45%, 50%, 60%, 70%, 80%, 90%, 100%, 2 fold, 3 fold, 4 fold, 5 fold, 6 fold, 7 fold, 8 fold, 9 fold, 10 fold or more.

### EXAMPLES

### Example 1: Rapalog-Mediated Inducible AAV Construct for the Expression of Inhibitory Nucleic Acids

This example describes an inducible AAV construct dependent on rapamycin/rapalog for the regulation of a promoter to direct the expression of miRNAs, shRNAs or small coding sequences. The single recombinant AAV vector approach to regulated gene silencing or gene expression with non-immunogenic human-based promoter elements should have broad impact and utility in the field of gene therapy.

AAV mediated RNAi is at the forefront in the gene therapy revolution to target dominant genetic diseases. Given the expanded role that AAV mediated gene therapy has filled using DNA directed transcription of artificial microRNAs as a modality to silence offending gene transcripts, it is imperative that a clinically compatible system for microRNA regulation be developed. Historically gene regulation with rAAV vectors has mostly relied on the use of the tet operon, a bacterial based system responding to the presence of doxycycline as the small molecule needed for gene regulation. This promoter system has proven effective for gene regulation both as the “tet on” and “tet off” versions. However, the largest roadblock to this system is its reliance on the expression of foreign proteins of bacterial origin, which has been shown to be targeted for elimination by the immune system. A mammalian derived, regulatable promoter based on the rapamycin inducible system is described here. This system is effective and immune compatible. The small size of the expressed miRNA transcript allows regulated expression with this in a single vector. A validated miRNA was used against human AAT which was shown with the single AAV vector to silence AAT after peripheral delivery in PiZ transgenic mice. Finally, the inducible activity of the regulated rAAV-miRNA was demonstrated against Huntington in a Huntington mouse model. Where a single injection of AAV led to silencing of Huntington protein after peripheral administration of the rapalog. This system can be of wide use to the field of AAV mediated RNAi for the treatment of disorder where silencing would want to be induce such as chronic pain by targeting sodium channels or other genes (e.g. Nav1.7, FAAH), or to treat hypercholesteremia by inducing the silencing of PCSK9. Induction of small aptamers to control other gene products could also benefit from this approach. Finally, induction of small genes (e.g. cytokines or small therapeutic peptide for cancer treatments) that can be accommodated within the single AAV cassette may also prove to be useful.

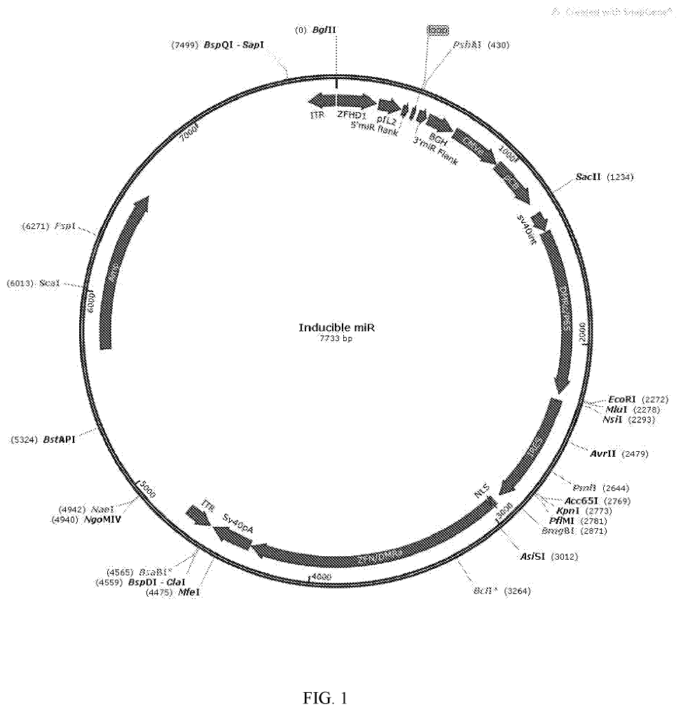

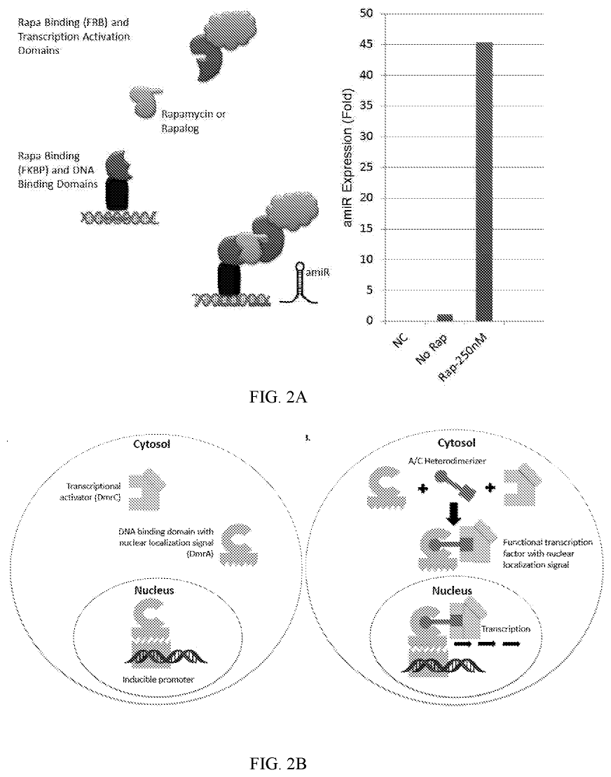

This approach relies on the heterodimerization of two different binding motifs that in the presence of a rapalog ligand complete the assembly of a transcriptional unit at the regulated promoter (see FIG. 2). The components are based on the human protein FKBP12 (FKBP, for FK506 binding protein) and its small molecule ligands. FKBP is an abundant cytoplasmic protein that serves as the initial intracellular target for FK506 and rapamycin. Rapamycin functions by binding with high affinity to FKBP, and then to the large PI3K homolog FRAP (RAFT, mTOR), thereby acting as a heterodimerizer to join the two proteins together. To use rapamycin to induce heterodimers transcription complex, the zinc finger (DNA binding domain) is fused to FKBP domains, and the p65 activation domain to a 93 amino acid portion of FRAP, termed FRB, that is sufficient for binding the FKBP-rapamycin complex (see FIG. 2). The system functions with rapamycin or the newly engineered rapalogs. The rapalogs have been chemically modified so that they no longer can bind to wild-type endogenous FRAP, greatly reducing immunosuppressive activity. An exemplary AAV construction depicting this design is shown in FIG. 1, having an nucleic acid sequence as set forth in SEQ ID NO: 3.

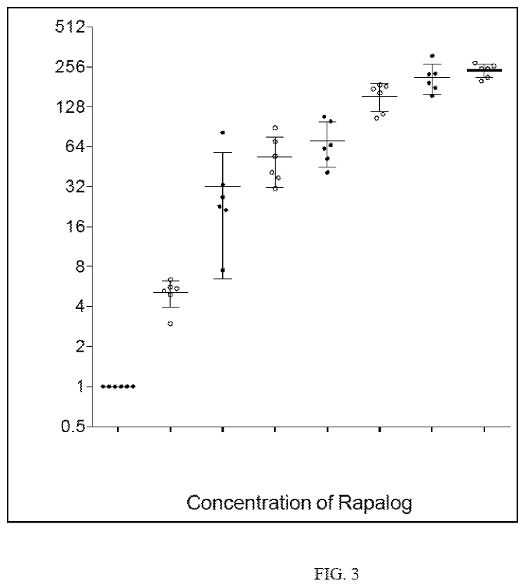

The preliminary data with the single pro-viral plasmid expressing all the necessary promoter elements, as well as the regulated promoter itself, demonstrates that it is feasible to regulate miRNA transcription. HEK-293 cells were transfected with the rAAV proviral plasmid expressing the regulated artificial miRNA (amiRNA) against AAT and either exposed to the rapalog or a PBS control solution. Forty-eight hours after rapalog induction the cells were harvested and a q-PCR assay for the mature form of the artificial miRNA was performed on the RNA fractions. As can be appreciated form the graph in FIG. 3, there is a 40-fold induction of the amiRNA when exposed to the rapalog. The levels of the amiRNA in the absence of the rapalog are near the limit of detection of the q-PRC assay.

### Example 2: Dynamic Concentration Range and Time Course of Rapalogs in Regulation of the Mir-RNA Expression

The dynamic concentration range of rapalog in regulating the miRNA expression was evaluated using an mir-RNA targeting human alpha-1-antitrypsin (AAT). The mir-RNA targeting against human alpha-1-antitrypsin (AAT) (miR-914) was cloned in the designated rapa-regulated mir-RNA vector. The resulting vector was transfected into HEK-293 cells to test the regulatory effect of the rapalogs by following procedures.

Transfection: 12-well plates with HEK-293 cell cultures were transfected with 0.5 μg combined rapa-regulated mir-AAT using Lipofectamine 3000. Same amount of sh-914 was also transfected as positive control.

Rapalog application: 24 hours after transfection, fresh medium with rapalogs was added to the cells every day. Various concentrations of rapalog (final concentration in medium) were tested.

Micro-RNA assay: Cells were harvested 48 hours after application of rapalogs to evaluate mir-914 expression level using ABI mircoRNA q-PCR assay.

Results of the experiments show that expression of mir-914 is very sensitive to the rapalog concentration from 32 nM to 500 nM (final concentration in medium). At 125 nM, the expression increased by about 50 folds over the basal expression, and at 500 nM it increased to over 200 folds. It seems the effects of rapalogs did not increase significantly above 500 nM. (FIG. 3)

Further, the time-effect of the rapalog in regulating miRNA expression was tested using the same vector described above.

Transfection: 12-well plates with HEK-293 cell cultures were transfected with 0.5 μg combined rapa-regulated mir-AAT (miR-914) using Lipofectamine 2000. Same amount of sh-914 was also transfected as positive control.

Rapalog application: 48 hours after transfection, wells were washed with PBS, change medium with Rapalog (500 nM final concentration in medium). Change the medium without rapalogs (removing of the rapalogs) 48 hours. Cells were harvested at 0, 24, 48, 72, and 96 hours after removing of the rapalogs.

Micro-RNA assay: using ABI mircoRNA q-PCR assay for human AAT to evaluate the mir-AAT to see the decrease.

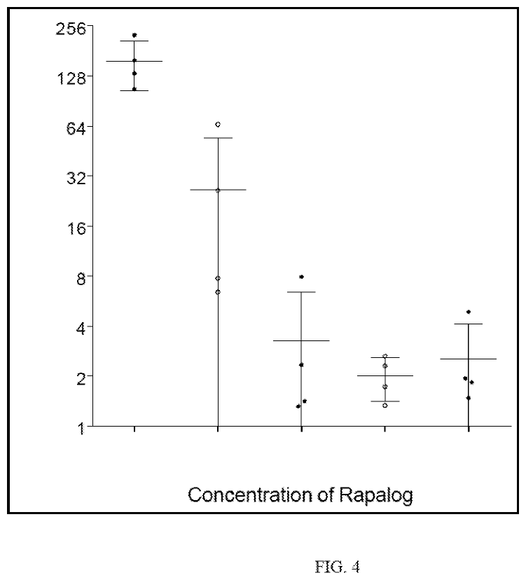

Results show that mir-AAT decrease with time. At about 3 days, the expression of mir-AAT reached to a very low level near the basal expression. (FIG. 4)

The rapa-regulated mir-AAT in vivo using AAT transgenic mice—PiZ transgenic mice. The injection of the AAV-inducible miR was performed according to the table below:

AAV-inducible miR
1 × 10¹², IP injection
Rapamycin:
15 ng/g body weight
(Intraperitoneal)
twice a week

Blood Sample were collected every two weeks.

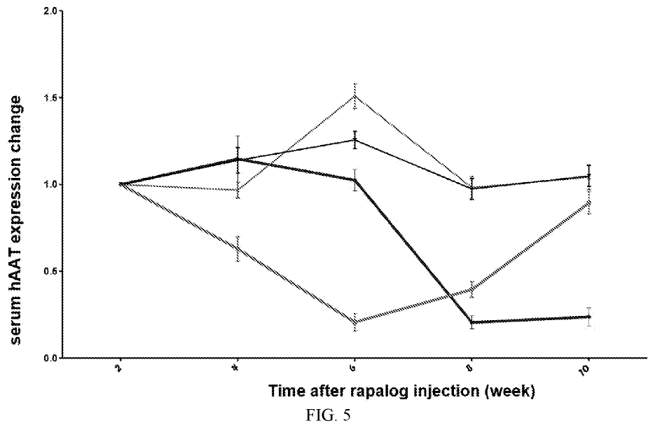

PiZ transgenic mice expressing human AAT either received and AAV expressing the inducible miR or PBS. Mice in groups 1 and 2 received the inducible miR against AAT. Serum levels of AAT were tracked every 2 weeks. Mice in group 1 were given rapamycin via IP injections starting on week 6 resulting in the lowering of serum AAT levels (FIG. 5). Mice in group 2 were given rapamycin at week 2 to induce AAT silencing and kept on the rapamycin injection every 2 weeks until week 8. As shown in FIG. 5, serum AAT levels in these mice responded to induction of miR expression as reflected by the lowering of serum AAT levels, these levels went back up at week 10 when the rapamycin was removed. Mice in group 3 were given rapamycin every 2 weeks to control for non-specific effects on AAT in sera levels. Mice in group 4 are mock and PBS controls.

### Example 3: Rapalog-Regulated miRNA Vector Against Human Huntingtin Gene (HTT)

The inducible activity of the regulated rAAV-miRNA was also demonstrated against Huntingtin gene in Huntington diseases in vitro and in vivo in Huntington mouse models(e.g., BAC97 and YAC128 mouse models). In the mouse models, a single injection of AAV led to silencing of Huntington protein after peripheral administration of the rapalog.

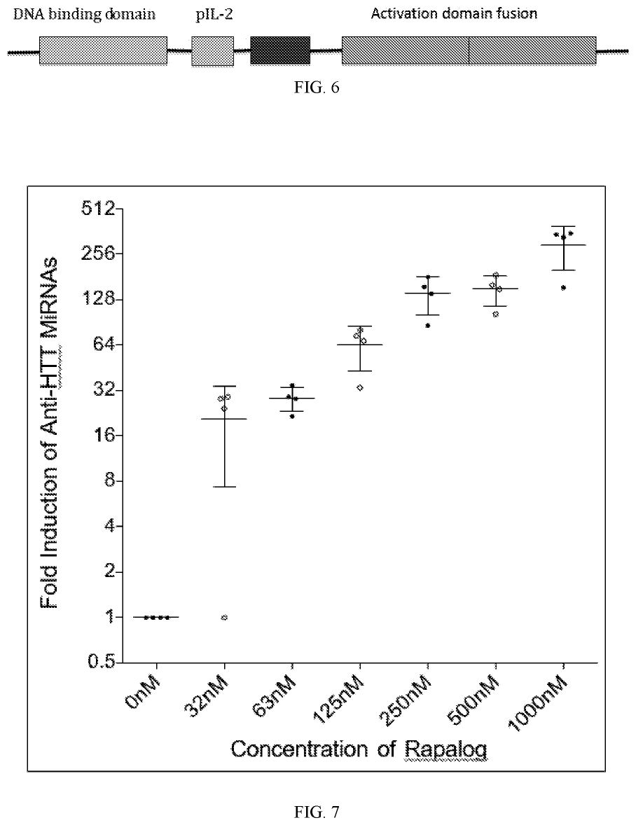

A mir-RNA targeting human huntingtin gene (HTT) 48 (position near 6433) was cloned into the designated rapa-regulated mir-RNA vector under the control of IL-2 promoter (FIG. 6). miR-6433-anti-HTT is encoded by a nucleic acid comprising the nucleotide sequence 5′-TAAGCATGGAGCTAGCAGGCT-3′ (SEQ ID NO: 5) The vector was transfected into HEK-293 cells to test the regulation effects on expression of the Mir-HTT6433.

Transfection: 12-well plates with HEK-293 cell cultures were transfected with 0.5 μg combined rapa-regulated mir-HTT6433 using Lipofectamine 2000.

Rapalog application: 24 hours after transfection, change medium with rapalogs (concentration series from 32 nM to 1 μM, final concentration in medium) daily.

Micro-RNA assay: Cells Harvest at 48 hours after application of rapalogs to evaluate the mir-HTT6433 using ABI mircoRNA q-PCR assay.

Similar to the previous experiments, results show that expression of the mir-HTT6433 is sensitive to the rapalog concentration from 32 nM to 500 nM (final concentration in Medium) (FIG. 7).

The Effects of rapalog-regulated mir-HTT6433 was further tested using a reporter vector: FfLuc-HTT-ex48. The firefly luciferase was fused to a partial sequence of HTT exon 48 such that the expression of the luciferase can be controlled by mir-HTT6433.

Co-transfection was performed of the firefly reporter and the rapalog-regulated mir-HTT6433 to test the effects of mir-RNA induced by rapalogs. Experiments are set as following:

Cells used: HEK-293, 96well plates.

Constructs: pAAV-ff-Luc-HTT-ex48, 0.02 μg/well Rapa-mir-HTT6433, 0.1 μg/well

Co-transfection: 0.1 μl Lipofectamine 2000/well

Twenty-four hours after co-transfection, cell culture medium was changed with fresh medium containing rapalogs in different concentration (from 32 nM to 1000 nM, final concentration in medium). Cell cultures were kept for incubation. Medium with rapalogs was changed daily.

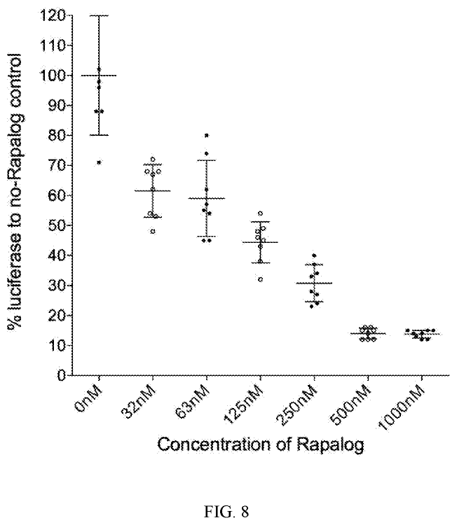

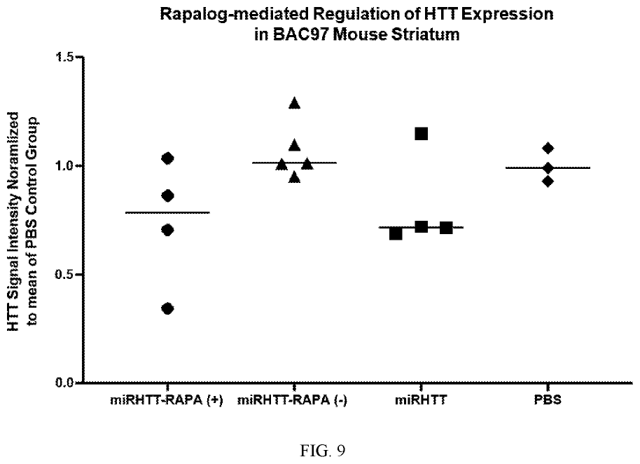

Cells were harvested at 24 hours, 48 hours and 72 hours for luciferase assay. From 48 hours after rapalog application, significant knock-down of the target reporter was observed. (FIG. 8).Further, Rapa-mir-HTT was tested in BAC97 mutant mice. In this experiment, heterozygous BAC97 mutant mice were intracranially injected at 11 weeks of age, with 1.35×10¹⁰ VP of AAV9-RAPA-miHTT. The mice received unilateral injections, directly into the striatum, with a total injection volume of 5.0 μL. The mice were housed for one month following surgeries to allow for AAV stability in the brain. At this point, the miRHTT-RAPA (+) treated mice started receiving rapalog three times per week intraperitoneally, at a dose of 15 mg/kg. The miRHTT-RAPA (−) mice were not treated with rapalog at any point. Two control groups were also injected at 11 weeks of age; one group with non-regulated AAV9-miRHTT and one group with PBS. All groups of mice were harvested six weeks after intracranial injections, regardless of rapalog treatment (FIG. 9). Brain punches were collected fresh and flash frozen, then sent for Western Blot assay. Human mutant HTT levels was measured by western blot assay after two weeks of rapalog regulation in AAV9-RAPA-miHTT treated BAC97 mice (RAPA+n=4), compared to AAV9-RAPA-miHTT treated BAC97 mice without rapalog-regulation (RAPA−n=5). A control group of BAC97 mice treated with non-regulated AAV9-miRHTT to compare efficacy with rapalog-mediated regulation (n=4). All groups are normalized to the PBS control group (n=3). The results indicated that human HTT protein expression was inhibited more in the rapalog treated group compared to the group of mice not receiving any rapalog in the striatal region on the injected side of the brain.

In addition, experiments were done using Huntington Disease mouse models to evaluate whether peripheral administration of small molecule would be able to cross the blood brain barrier and regulate AAV-miRNA expression in the CNS.

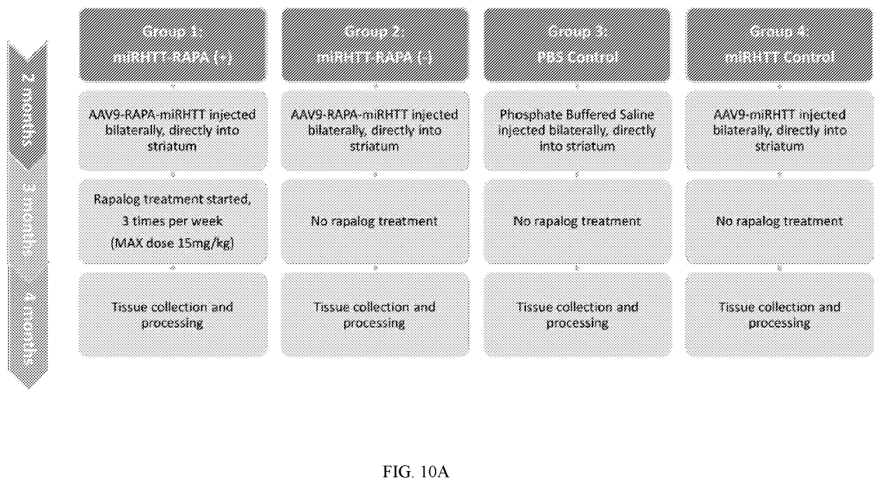

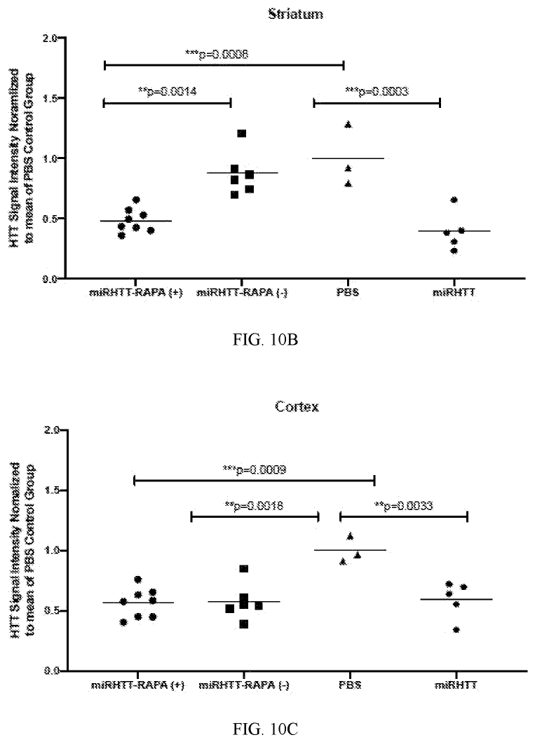

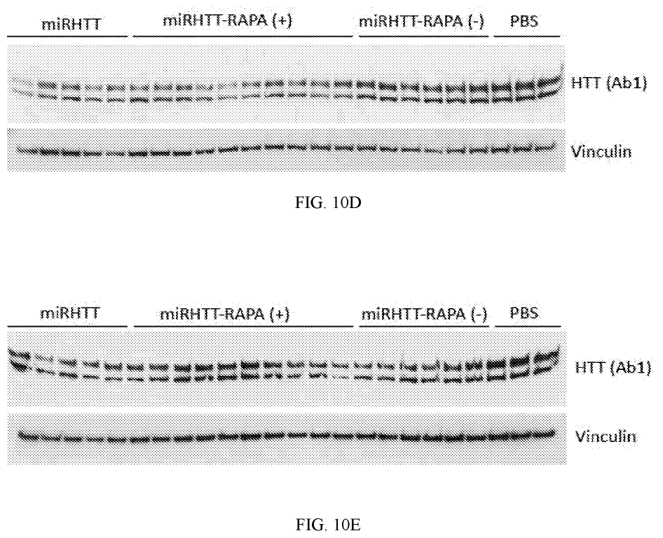

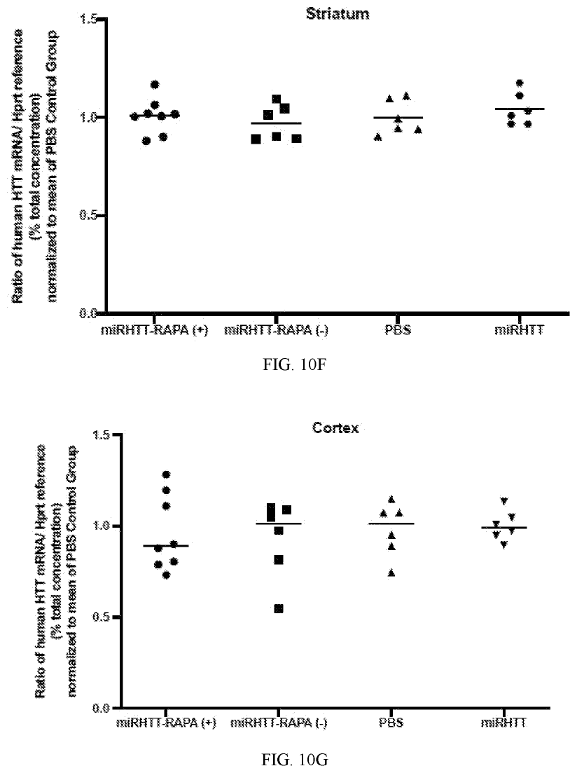

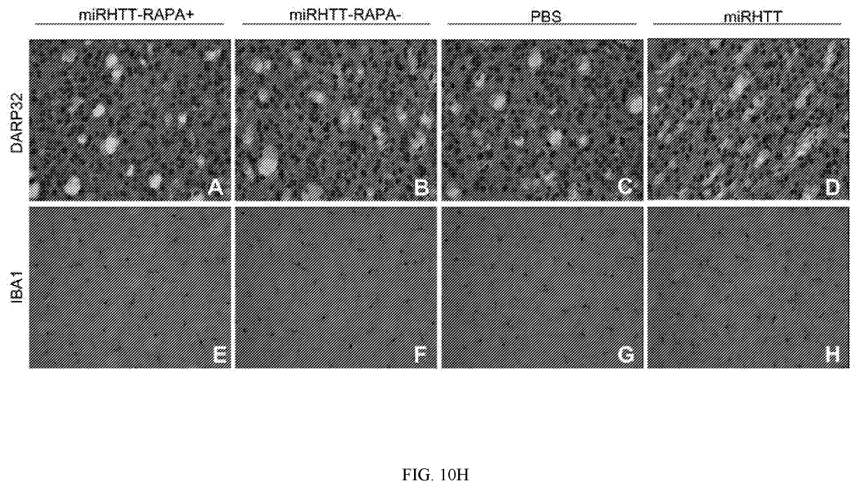

Rapa-mir-HTT was further tested in a different Huntington Disease mouse model—YAC128-HD mouse model. The timeline and groups of mice in this experiments was illustrated in FIGS. 10A-10H. The time-course and groups of mice used was described in FIG. 10A. Mice in Group 1 and Group 2 were injected with AAV9-RAPA-miRHTT directly into striatum bilaterally at 2 months old. Mice in Group 3 were included as PBS control. Mice in Group 4 were injected with AAV9-miRHTT directly into striatum bilaterally at 2 months old as positive control. Group 1 was then treated with rapalog orally 3 times a week at a maximum dose of 15 mg/kg, which Groups 2-4 did not receive any rapalog treatment. The mice were sacrificed, and striatum and cortex tissues were collected and processed for evaluation of HTT expression. Human mutant HTT levels detected by western blot assay after one month of rapalog-mediated regulation in AAV9-RAPA-miHTT treated YAC128 mice (Group 1, n=8), compared to AAV9-RAPA-miHTT treated YAC128 mice without rapalog-regulation (Group 2, n=5). A control group of YAC128 mice treated with non-regulated AAV9-miRHTT to compare efficacy with rapalog-mediated regulation (Group 4, n=6). All groups are normalized to the PBS control group (Group 3, n=3). Results are shown for the striatal (FIG. 5B) and cortex (FIG. 5C) regions on one side of the brain. The graphs show distribution of individual animals and means (horizontal bars) for each treatment group. Significance values are based on One-way ANOVA statistical analysis. Striatal (FIG. 5D) and cortex (FIG. 5E) western blot images show mutant HTT detected with AB1 antibody and Vinculin as a control. The results show that rapalog dosing causes a significant reduction in human HTT protein expression in the brain. In contrast, in the absence of rapalog, the AAV is inactive and HTT protein levels in the brain are unchanged. This work demonstrates that an artificial miRNA targeting huntingtin can be regulated in vivo. It provides a tool for further investigation of HTT gene silencing and could be used to mitigate concerns about long-term huntingtin silencing. Further, brain silencing of HTT in the mice were achieved after oral administration of rapalog, solidifying the data that the rapalog's capability of crossing the blood brain barrier and inducing gene silencing in the CNS.

Further, Human mutant HTT mRNA levels in the striatum and cortex in each group was measured by Droplet Digital PCR (DD PCR). Using the High Capacity RT Kit (Thermo Fischer Scientific), 50 ng of RNA was used to synthesize cDNA for DD PCR. Equal amounts were added for each reaction and all samples we normalized to HPRT reference probe. The graph shows distribution of individual animals and means (horizontal bars) for each treatment group. No Significant differences were found based on One-way ANOVA statistical analysis.

Additionally, Immunohistochemistry stain was used to verify the safety of AAV vector injections. Brain sections were stained with DARP32 (images A-D) and IBA1 (images E-H) on one side of the brain. Every 5th brain section was analyzed, and pictures were taken in the center of the striatum (at 20× magnification) with a Nikon Eclipse E600 microscope and a Nikon-Qi1MC camera with NIS-Elements (FIG. 10H).

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. An isolated nucleic acid comprising:
(i) a first expression cassette comprising a regulatable promoter operably linked to a first transgene; and
(ii) a second expression cassette comprising second promoter operably linked to a second transgene, wherein the second transgene encodes a FKBP-rapamycin binding protein (FRB), a transcription activator domain, a rapamycin-binding protein (FKBP), and a DNA binding protein that specifically binds to a portion of the regulatable promoter of (i).

2. The isolated nucleic acid of claim 1, wherein the regulatable promoter is a weak promoter, optionally an IL-2 promoter.

3. (canceled)

4. The isolated nucleic acid of claim 1, wherein the first transgene comprises a nucleotide sequence encoding an inhibitory nucleic acid, a transfer RNA (tRNA), a guide RNA, or an aptamer.

5. The isolated nucleic acid of claim 4, wherein the inhibitory nucleic acid is an inhibitory RNA selected from the group consisting of a microRNA, a small interfering RNA (siRNA), a short hairpin RNA, an artificial miRNA (AmiRNA), or an antagomir.

6. The isolated nucleic acid of claim 5, wherein the inhibitory RNA is an inhibitory RNA targeting alpha-1-antitrypsin (AAT), human huntingtin gene (HTT), Nav1.7, FAAH, or PCSK9.

7-10. (canceled)

11. The isolated nucleic acid of claim 1, wherein the second promoter is a constitutive promoter, an inducible promoter, or a tissue specific promoter optionally wherein the second promoter is a minimal promoter.

12. (canceled)

13. The isolated nucleic acid of claim 1, wherein the FKBP is FKBP12 or wherein the DNA binding domain is a zinc finger domain or a dCas protein.

14-18. (canceled)

19. The isolated nucleic acid of claim 1, wherein the FRB domain is a FKBP12-rapamycin-associated protein (FRAP) domain or wherein the transcription activator domain is p65 activation domain, VP4, or VP16.

20-25. (canceled)

26. The isolated nucleic acid of claim 1, wherein the first and/or the second transgene each further comprises a 3′ untranslated region (3′UTR) or a one or more miRNA binding sites.

27-31. (canceled)

32. The isolated nucleic acid of claim 1, further comprising two flanking long terminal repeats (LTRs).

33. The isolated nucleic acid of claim 1, further comprising two flanking adeno-associated virus inverted terminal repeats (ITRs).

34. The isolated nucleic acid of claim 33, wherein the ITRs are adeno-associated virus ITRs of a serotype selected from the group consisting of AAV1 ITR, AAV2 ITR, AAV3 ITR, AAV4 ITR, AAV5 ITR, and AAV6 ITR.

35-38. (canceled)

39. A recombinant adeno-associated virus (rAAV) vector comprising, in 5′ to 3′ order:
(a) a 5′ AAV ITR;
(b) a regulatable promoter;
(c) a first transgene;
(d) a second promoter;
(e) a second transgene comprising a nucleotide sequence encoding a FRB-p65 fusion protein, a nucleotide sequence encoding an internal ribosome entry site (IRES), a nucleotide sequence encoding a FKBP-zinc finder domain fusion protein; and
(f) a 3′ AAV ITR.

40. A recombinant lentivirus comprising a lentiviral capsid containing the isolated nucleic acid of claim 1.

41. A recombinant adeno-associated virus (rAAV) comprising:
(i) an isolated nucleic acid of claim 1; and
(ii) at least one AAV capsid protein.

42. The rAAV of claim 41, wherein the capsid protein is of a serotype selected from AAV1, AAV2, AAV3, AAV4, AAV5, AAV6, AAV7, AAV8, AAV9 and a variant of any of the foregoing.

43. The rAAV of claim 41 or 42, wherein the rAAV is a single-stranded AAV (ssAAV).

44. A recombinant adeno-associated virus (rAAV), comprising:
(i) an AAV capsid protein; and
(ii) an isolated nucleic acid comprising, in 5′ to 3′ order:
(a) a 5′ AAV ITR;
(b) a regulatable promoter;
(c) a first transgene;
(d) a second promoter;
(e) a second transgene comprising a nucleotide sequence encoding a FRB-p65 fusion protein, a nucleotide sequence encoding an internal ribosome entry site (IRES), a nucleotide sequence encoding a FKBP-zinc finder domain fusion protein; and
(f) a 3′ AAV ITR.

45-51. (canceled)

52. A method for treating a disease in a subject in need thereof, the method comprising:
(i) administering to the subject a therapeutically effective amount of the isolated nucleic acid of claim 1; and
(ii) administering to the subject an effective amount of rapamycin or a rapalog.

53-67. (canceled)

68. A method for modulating transgene expression in a subject, the method comprising:
(i) administering the isolated nucleic acid claim 1, and a rapalog to a subject;
(ii) measuring expression of transgene in the subject relative to a control expression level of the first transgene;
(iii) adjusting the dose of rapalog-based on expression level measured in (ii), wherein if the expression level measured in (ii) is increased relative to the control level, administering the same or less concentration of the rapalog; and wherein if the expression level measured in (ii) is the same or decreased relative to the control level, administering a higher concentration of the rapalog to the subject.
